(19) United States
(12) Patent Application Publication
(10) Pub. No.: US 2024/0051975 A1
(43) Pub. Date: Feb. 15, 2024

(54) NOVEL NAMPT ENZYME AGONIST AND PREPARATION AND USE THEREOF

(71) Applicant: TSINGHUA UNIVERSITY, Beijing (CN)

(72) Inventors: Gelin WANG, Beijing (CN); Yefeng TANG, Beijing (CN); Hong YAO, Beijing (CN); Minghui LIU, Beijing (CN); Leibo WANG, Beijing (CN); Chenyu LI, Beijing (CN); Ruoxi ZHANG, Beijing (CN); Yumeng ZU, Beijing (CN); Chuo WU, Beijing (CN); Feifei LI, Beijing (CN); Shuangquan CHEN, Beijing (CN)

(21) Appl. No.: 18/258,346

(22) PCT Filed: Feb. 14, 2022
(86) PCT No.: PCT/CN2022/076187

§ 371 Date: Sep. 5, 2023

(30) Foreign Application Priority Data
Dec. 22, 2020 (CN) 202011525254.7

(51) Int. Cl. C07F 5/02; C07C 235/24; C07D 213/65; C07C 255/50; C07C 311/16; C07C 279/04; A61P 25/28

(52) U.S. Cl. CPC C07F 5/025; C07C 235/24; C07D 213/65; C07C 255/50; C07C 311/16; C07C 279/04; A61P 25/28; C07C 2601/16

(57) ABSTRACT

The present invention provides a class of novel NAMPT enzyme agonist and preparation and use thereof, which has a structural formula as shown in formula I or formula II. The present invention screens the NAMPT agonist NAT from the chemical small molecule library, and the NAT exhibits a good cytoprotective effect and a good anti-neurodegeneration effect in animal models of neurodegeneration. We studied the binding of NAT to enzymes, and then carried out multiple rounds of structure optimization based on the chemical structure characteristics of NAT and its enzyme activity properties, and obtained a relatively defined structure-activity relationship. The present patent not only lays the foundation for developing innovative drugs for anti-aging and neurodegenerative diseases, but also theoretically provides a proof-of-concept that enhancing NAMPT enzyme activity plays an important role in neuroprotection.

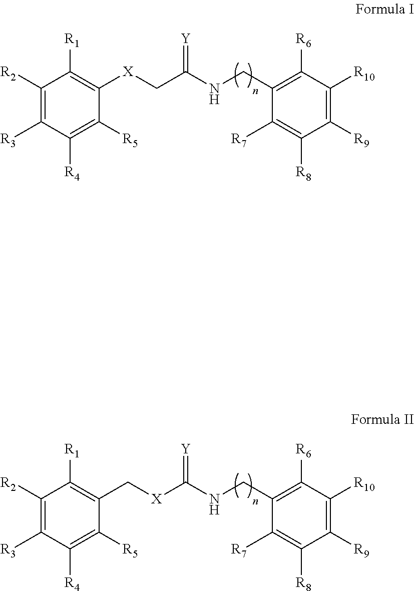

### RELATED APPLICATIONS

The present patent application is a U.S. National Phase of International Application Number PCT/CN2022/076187 filed Feb. 14, 2022, and claims the priority of the Chinese patent application entitled “Novel NAMPT Enzyme Agonist and Preparation and Use Thereof” with application number 202011525254.7 and application date of Dec. 22, 2020. All content stated in this priority text is cited or incorporated or included into the present patent application.

### FIELD OF THE INVENTION

The present invention relates to the fields of medicinal chemistry, enzymology and pharmacology, and specifically to a novel NAMPT enzyme agonist and preparation and use thereof.

### BACKGROUND OF THE INVENTION

1. Pharmaceutical Importance and Challenges of Drugs for Anti-Aging and Neurodegenerative Disease

Aging is a rather complex process, and in recent years, there has been a significant global rise in aging-related diseases, particularly neurodegenerative diseases. The refractory nature of neurodegenerative diseases and the need for special care for patients place a heavy burden on families, societies and countries. It has become a daunting task to effectively control, delay aging and treat neurodegenerative diseases.

Despite the rising incidence of neurodegenerative diseases, due to the lack of a deep understanding of the cause of the disease and the mechanism of disease development, the research and development of specific new drugs have repeatedly failed, and the progress is extremely limited. The currently marketed drugs can only relieve early symptoms and cannot prevent the development of the disease, and there is still no drug that can effectively curb the development of the disease.

2. Strategic Advantages of Targeting NAD Metabolism Anti-Aging and Neurodegeneration

Nicotinamide adenine dinucleotide (NAD) is one of the central metabolites controlling a variety of biological processes including energy metabolism and signal transduction. NAD can be synthesized de novo from tryptophan, or it can be synthesized through a salvage pathway from nicotinamide (NAM), nicotinic acid (NA), and nicotinamide riboside (NR) (J. Preiss, P. Handler, Biosynthesis of diphosphopyridine nucleotide. I. Identification of intermediates. J Biol Chem 233, 488-492 (1958)). Among these pathways, mammals mainly use the salvage pathway derived from NAM as the main source of NAD in vivo. In this process, the first step is catalyzed by nicotinamide phosphoribosyltransferase (NAMPT), that synthesizes nicotinamide mononucleotide (NMN) from NAM and phosphoribosyl pyrophosphate (PRPP); the second step is catalyzed by nicotinamide mononucleotide adenylyltransferase. (NMNAT) to further synthesize NAD (K. L. Bogan, C. Brenner, Nicotinic acid, nicotinamide, and nicotinamide riboside: a molecular evaluation of NAD precursor vitamins in human nutrition. Annu Rev Nutr 28, 115-130 (2008).). NAMPT is the rate-limiting enzyme in this NAD biosynthesis pathway, and its activity is essential for maintaining stable intracellular NAD levels (J. R. Revollo, A. A. Grimm, S Imai, The NAD biosynthesis pathway mediated by nicotinamide phosphoribosyltransferase regplates Sir2 activity in mammalian cells. J Biol Chem 279, 50754-50763 (2004); O. Stromland et al., Keeping the balance in NAD metabolism. Biochem Soc Trans, (2019).). There have been many exciting recent discoveries demonstrating that NAMPT and NAD play an important role in a variety of important physiological processes in the body, such as energy metabolism, adaptive stress response, cell death, stem cell proliferation and self-renewal, and inflammation. Therefore, dysregulation of NAD metabolism is also associated with many diseases, including neurodegenerative diseases, cardiovascular diseases, metabolic syndrome, cancer, infectious diseases, inflammation, aging and many other diseases. Supplementing NAD synthesis precursors or activating NAMPT can be of great benefit in delaying the development of the above diseases (A. Garten et al., Physiological and pathophysiological roles of NAMPT and NAD metabolism. Nat Rev Endocrinol 11, 535-546 (2015) ; Y Yang, A. A. Sauve, NAD(+) metabolism: Bioenergetics, signaling and manipulation for therapy. Biochim Biophys Acta 1864, 1787-1800 (2016)). Therefore, increasing intracellular NAD is expected to become a novel therapeutic approach to prevent and treat aging-related complex diseases in general. At present, the enhancement of NAD is mainly achieved by supplying NAD precursors such as NR, NMN or NAM. These NAD precursors protect against a variety of aging-related diseases in animal models and boost immunity, promote blood flow, and protect tissues and organs from disease and damage. In recent years, there have been more than ten clinical trials in progress, but the dose of NAD precursor needs to be taken is very large, further studies on pharmacokinetics and safety are needed, and the results of clinical trials have not yet been published (E. Verdin, NAD(+) in aging, metabolism, and neurodegeneration. Science 350, 1208-1213 (2015).; H. zhang et al., NAD (+) Repleting Improves Mitochondric and Stem Cell Funct ION and Enhances Life Span in Mice. Science 352, 1436-1443 (2016); G. Wang et al., P7C3 neuroprotective chemicals function by activating the rate-limiting enzyme in NAD salt. Cell 158, 1324-1334 (2014)).

### TECHNICAL PROBLEM

Very few NAMPT agonists have been reported in the prior art, which are far from meeting the clinical needs, and there is an urgent need to develop new small-molecule NAMPT agonists.

### TECHNICAL SOLUTION

One object of the present invention is to provide a class of aromatic compounds with NAMPT activation effect, namely NAMPT enzyme agonists.

The aromatic compound with NAMPT activation effect provided by the present invention, that is, the NAMPT enzyme agonist, has a structural formula as shown in formula I or formula II:

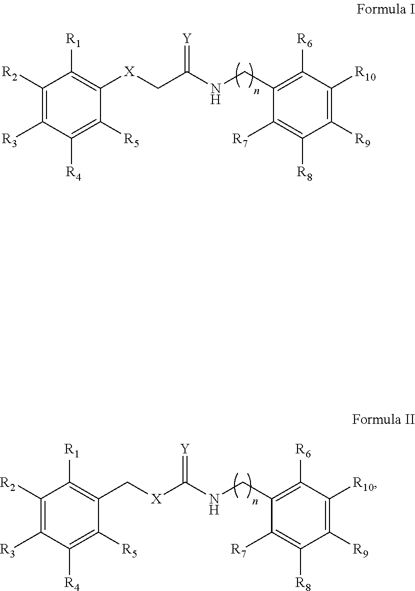

in formula I and formula II, X represents O or NH;
Y represents O;
n is 0 or 1;
in the formula, R₁, R₂, R₃, R₄, R₅, R₆, R₇, R₈, R₉, R₁₀ each independently represent H, C1-C6 straight or branched chain alkyl (such as methyl, ethyl, isopropyl, tert-butyl), C3-C6 cycloalkyl (such as cyclopropyl, cyclobutyl, cyclopentyl), halogen substituted C1-C6 straight or branched chain alkyl (such as trifluoromethyl), hydroxyl, mercapto, halogen, cyano, nitro, boronic acid group, boron ester group, carboxyl, ester (such as —COOEt. —COOCH₃), carbonyl (such as —COCH₃), phenoxy (such as OPh), amidino (such as —CNHNH₂), amido (such as —CONH₂), imide, sulfanilamide, pyrazolyl, substituted or unsubstituted amino, substituted or unsubstituted morpholinyl, substituted or unsubstituted piperidyl, substituted or unsubstituted piperazinyl, substituted or unsubstituted phenyl, substituted or unsubstituted pyridyl, substituted or unsubstituted C1-C6 alkoxy, substituted or unsubstituted C1-C4 alkyl hydroxyl, substituted or unsubstituted C1-C4 alkyl morpholinyl, substituted or unsubstituted C1-C4 alkyl piperidinyl, substituted or unsubstituted C1-C4 alkyl piperazinyl;
the substituted amino means that at least one H on the amino is substituted by C1-C6 alkyl or tert-butoxycarbonyl (Boc);
the substituted morpholinyl means that one or more carbons on the morpholinyl are substituted by the following groups: hydroxyl, C1-C6 alkyl, C1-C6 alkoxy, halogen, halogen substituted C1-C6 alkyl (such as trifluoromethyl), nitro, cyano, amino or substituted amino;
the substituted piperidinyl means that one or more carbons on the piperidinyl are substituted by the following groups: hydroxyl, C1-C6 alkyl, C1-C6 alkoxy, halogen, halogen substituted C1-C6 alkyl (such as trifluoromethyl), nitro, cyano, amino or substituted amino;
the substituted piperazinyl means that one or more carbons on the piperazinyl are substituted by the following groups: hydroxyl, C1-C6 alkyl, C1-C6 alkoxy, halogen, halogen substituted C1-C6 alkyl (such as trifluoromethyl), nitro, cyano, amino or substituted amino, and can also be that the H on the N of piperazinyl is substituted by the following groups: unsubstituted C1-C6 alkyl or substituted C1-C6 alkyl, unsubstituted C1-C6 alkoxy or substituted C1-C6 alkoxy, acyl;
the substituted C1-C6 alkyl means that one or more hydrogens on the unsubstituted C1-C6 alkyl are substituted by hydroxyl, halogen, nitro, cyano, amino, unsubstituted phenyl or substituted phenyl (such as —CHOHCH₃);
the unsubstituted C1-C4 alkoxy is selected from a group consisting of methoxy, ethoxy, n-propoxy, isopropoxy, n-butoxy, isobutoxy, or tert-butoxy; the substituted C1-C4 alkoxy means that one or more hydrogens on the unsubstituted C1-C4 alkoxy are substituted by hydroxyl, halogen, nitro, cyano, amino, phenyl or substituted phenyl, and can also mean that one or more carbons on the unsubstituted C1-C4 alkoxy are substituted by O, N;
the substituted phenyl means that one or more hydrogens on the benzene ring are substituted by the following groups: hydroxyl, unsubstituted C1-C4 alkyl or substituted C1-C4 alkyl, unsubstituted C1-C4 alkoxy or substituted C1-C4 alkoxy, halogen, nitro, cyano, amino;
the unsubstituted pyridyl means that the connection position is on different carbons of the pyridyl, such as 2-pyridyl, 3-pyridyl, 4-pyridyl; the substituted pyridyl means that one or more carbons on the pyridyl are substituted by the following groups: hydroxyl, C1-C4 alkyl, C1-C4 alkoxy, halogen, halogen substituted 1C-6C alkyl (such as trifluoromethyl), nitro, cyano, amino or substituted amino;
the unsubstituted C1-C4 alkylamino is selected from a group consisting of methylamino, ethylamino, n-propylamino, isopropylamino, formylamino, acetamido, formimidoamino, cyclopropylamino, cyclobutylamino, cyclopentylamino, cyclohexylamino; the substituted C1-C4 alkylamino means that one or more hydrogens on the alkyl of unsubstituted C1-C4 alkylamino are substituted by hydroxyl, halogen, cyano, amino, phenyl or substituted phenyl, and can also mean that one or more carbons on the alkyl of unsubstituted C1-C4 alkylamino are substituted by O, N, and can also mean that one or more H on N of the unsubstituted C1-C4 alkylamino is substituted by methyl, ethyl, formyl, acetyl, cyclopropylamino, cyclobutylamino, cyclopentylamino, cyclohexylamino;
the unsubstituted C1-C4 alkyl hydroxyl means methyl hydroxyl, ethyl hydroxyl, n-propyl hydroxyl, isopropyl hydroxyl, n-butyl hydroxyl, isobutyl hydroxyl, tert-butyl hydroxyl; the substituted C1-C4 alkyl hydroxyl means that one or more hydrogens on the alkyl of unsubstituted C1-C4 alkyl hydroxyl are substituted by hydroxyl, halogen, cyano, amino, phenyl or substituted phenyl, and can also means that one or more carbons on the alkyl of the unsubstituted C1-C4 alkyl hydroxyl are replaced by O, N;
the unsubstituted C1-C4 alkyl morpholinyl means that methyl morpholinyl, ethyl morpholinyl, n-propyl morpholinyl, isopropyl morpholinyl, n-butyl morpholinyl, isobutyl morpholinyl, tert-butyl morpholinyl; the substituted C1-C4 alkyl morpholinyl means that one or more hydrogens on the alkyl of the unsubstituted C1-C4 alkyl morpholinyl are substituted by hydroxyl, halogen, cyano, amino, phenyl or substituted phenyl, and can also mean that one or more carbons on the alkyl of unsubstituted C1-C4 alkyl morpholinyl are substituted by O, N;
the unsubstituted C1-C4 alkyl piperidinyl means that methyl piperidinyl, ethyl piperidinyl, n-propyl piperidinyl, isopropyl piperidinyl, n-butyl piperidinyl, isobutyl piperidinyl, tert-butyl piperidinyl; the substituted C1-C4 alkyl piperidinyl means that one or more hydrogens on the alkyl of unsubstituted C1-C4 alkyl piperidinyl are substituted by hydroxyl, halogen, cyano, amino, phenyl or substituted phenyl;
the unsubstituted C1-C4 alkyl piperazinyl means that methyl piperazinyl, ethyl piperazinyl, n-propyl piperazinyl, isopropyl piperazinyl, n-butyl piperazinyl, isobutyl piperazinyl, tert-butyl piperazinyl; the substituted C1-C4 alkyl piperazinyl means that one or more hydrogens on the alkyl of unsubstituted C1-C4 alkyl piperazinyl are replaced by hydroxyl, halogen, cyano, amino, phenyl or substituted phenyl, and can also mean that the H on the N of unsubstituted C1-C4 alkyl piperazinyl are substituted by the following groups: unsubstituted C1-C4 alkyl or substituted C1-C4 alkyl, unsubstituted C1-C4 alkoxy or substituted C1-C4 alkoxy, acyl;
the substituted amino is selected from a group consisting of methylamino, dimethylamino, ethylamino, diethylamino, n-propylamino, di-n-propylamino, isopropylamino, diisopropylamino, formylamino, acetylamino, formimidoamino, cyclopropylamino, cyclobutylamino, cyclopentylamino, cyclohexylamino, and can also mean that azacyclobutyl, azacyclopentyl, azacyclohexyl, 2-oxo-azacyclobutyl, 2-oxo-azacyclopentyl, 2-oxo-azacyclohexyl;
the benzene ring in formula I or formula II can also be substituted by other aromatic rings, the other aromatic rings can be pyridine rings, naphthalene rings, furan rings, pyrrole rings, quinoline rings, etc.

The present invention further provides pharmaceutically acceptable salts of aromatic compounds represented by formula I or formula II, such as inorganic acid salts such as hydrochloride, sulfate, hydrobromide or phosphate salts of the aromatic compounds; it can also be organic acid salts such as oxalate, maleate, benzoate or fumarate of the aromatic compound.

Another object of the present invention is to provide a method for the synthesis of the above-mentioned aromatic compound with NAMPT activation effect, namely, the compound represented by formula I and formula II.

The method for the synthesis of the aromatic compound (compound represented by formula I) with NAMPT activation effect provided by the present invention comprises:
(1) reacting the compound shown in formula III with tert-butyl bromoacetate to obtain compound shown in formula IV, followed by the compound shown in formula IV in the presence of trifluoroacetic acid (TFA) to obtain the compound shown in formula V;

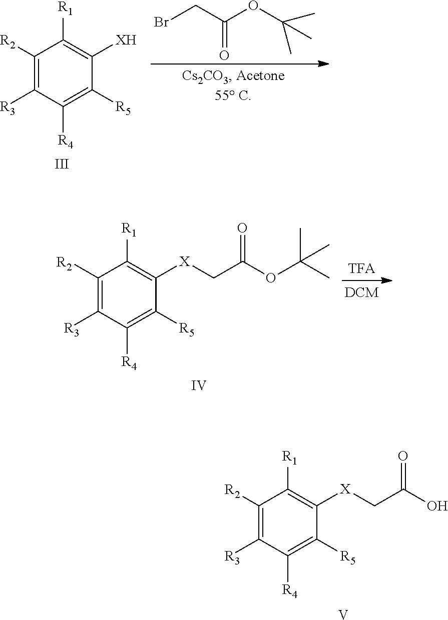

the definitions of R₁, R₂, R₃, R₄, R₅, X in formulas III, IV and V are the same as formula I;
(2a) reacting the compound shown in formula V with oxalyl chloride (COCl)₂ to obtain the compound shown in formula VI, and then reacting the compound shown in formula VI with the compound shown in formula VII to obtain formula I;

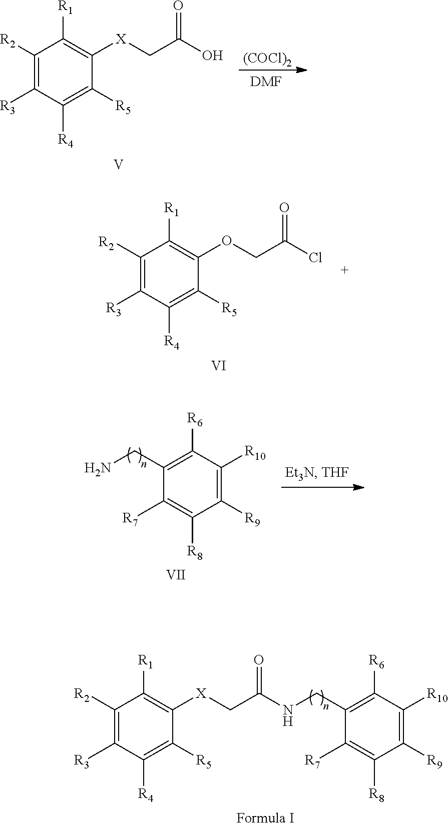

the definitions of R₁, R₂, R₃, R₄, R₅, R₆, R₇, R₈, R₉, R₁₀, X, n in formula VI and VII are the same as formula I;
or
(2b) formula V and formula VII are directly condensed under the conditions of 1-ethyl-(3-dimethylaminopropyl) carbodiimide hydrochloride(EDCI) and 1 -hydroxybenzotriazole(HOBt) to obtain formula I;

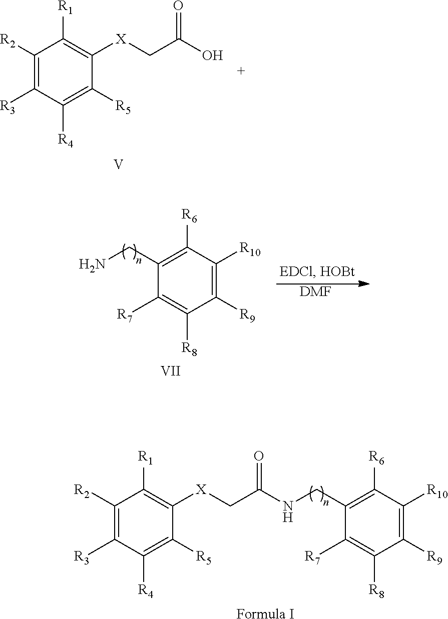

The method for the synthesis of the aromatic compound (compound represented by formula II) with NAMPT activation effect provided by the present invention comprises:
(1a) reacting formula X with formula XI to obtain formula XII, and then reacting formula XII with formula VIII to obtain formula II;

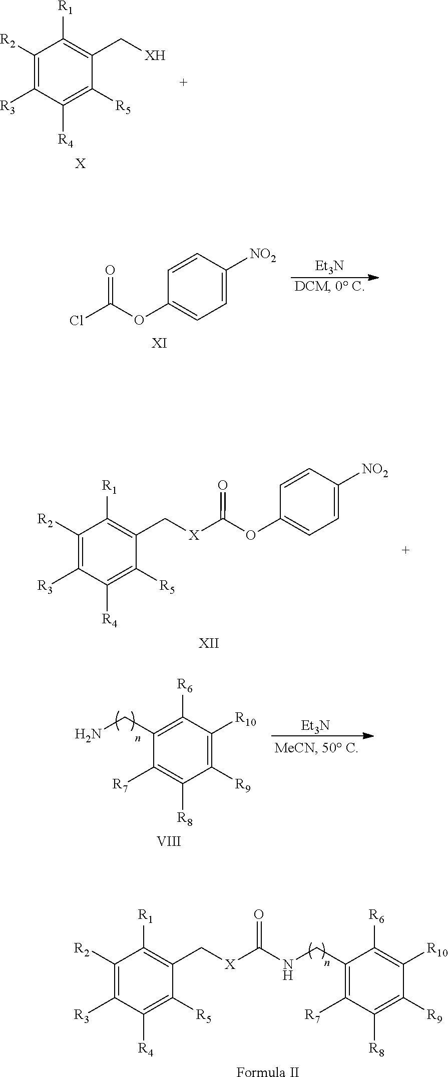

the definitions of R₁, R₂, R₃, R₄, R₅, R₆, R₇, R₈, R₉, R₁₀, X in formula X, XI, XII are the same as formula II;

in VIII, the definitions of R₆, R₇, R₅, R₉, R₁₀ and n are the same as formula II;

or,
(1b) reacting formula X with formula XV to obtain formula II;

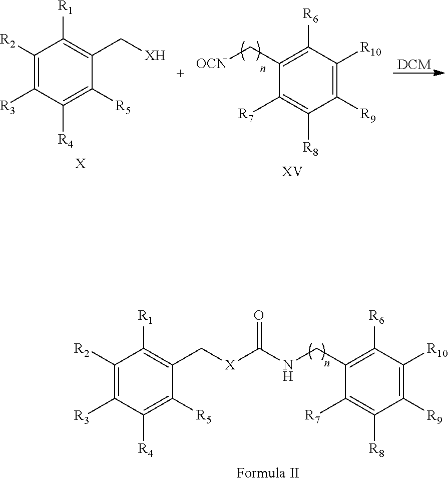

the definitions of R₆, R₇, R₅, R₉, R₁₀, n in formula XV are the same as formula II.

Another object of the present invention is to provide the use of the above-mentioned aromatic compound with NAMPT activation effect in the preparation of products for anti-aging and treatment of neurodegenerative diseases.

Specifically, herein the neurodegenerative disease is chemotherapeutic drug-induced peripheral neuropathy (CIPN).

The present invention further provides a drug for treating neurodegenerative diseases or anti-aging, the active ingredient of which is an aromatic compound represented by formula I or formula II or a pharmaceutically acceptable salt thereof.

### BENEFICIAL EFFECTS

The preparation method provided by the present invention starts from simple and easy-to-obtain raw materials, and the aromatic compound represented by formula I or formula II can be obtained through 4 to 5 steps of reaction; the aromatic compound provided by the present invention has a good NAMPT-activation effect.

The present invention screens the NAMPT agonist NAT from the chemical small molecule library, and the NAT exhibits a good cytoprotective effect and a good anti-neurodegeneration effect in animal models of neurodegeneration. We studied the binding of NAT to enzymes, and then carried out multiple rounds of structure optimization according to the chemical structure characteristics of NAT and enzyme activity properties, and obtained a relatively clear structure-activity relationship. The present patent not only lays the foundation for providing innovative drugs for anti-aging and neurodegenerative diseases, but also theoretically provides a proof-of-concept that enhancing NAMPT enzyme activity plays an important role in neuroprotection.

### BRIEF DESCRIPTION OF THE FIGURES

FIG. 1 shows that NAT increases the enzyme reaction rate of NAMPT.

FIG. 2 shows the binding curve of NAT to NAMPT determined by an ITC method.

FIG. 3 shows a schematic diagram of the method for calculating the enzyme activation activity of NAT and derivatives thereof.

FIGS. 4A and 4B show the binding curve of NAT derivatives to NAMPT determined by an ITC method. FIG. 4A shows the binding of compound 2 to NAMPT, and FIG. 4B shows the binding of compound 21 to NAMPT.

FIGS. 5A and 5B show the biological activity of NAT and derivatives thereof. FIG. 5A shows the enzyme activation activity, and FIG. 5B shows the cytoprotective activity.

FIG. 6 shows the correlation between the enzyme activation activity and cytoprotective activity of NAT and derivatives thereof.

FIG. 7 shows a mouse model of CIPN.

FIG. 8 shows the neuroprotective effect of NAT in the mouse model of CIPN.

### EMBODIMENTS OF THE PRESENT INVENTION

The structures of the compounds in the following examples are shown in Table 1, and the example numbers are the same as the compound numbers.

The present invention will be further described below in conjunction with the examples, but the present invention is not limited in any way, and any changes or improvements made based on the guidance of the present invention belong to the protection scope of the present invention.

The ¹H and ¹³C NMR spectra in the following examples are all measured with a Bruker AM-400 NMR instrument, and the hydrogen spectrum is measured at 400.0 MHz, and the carbon spectrum is measured at 100.6 MHz. Chemical shifts are corrected by TMS signal in CDCl₃. HR-ESI-MS data are determined by Bruker Apex IV FTMS.

The experimental methods used in the following examples are conventional methods unless otherwise specified.

The materials and reagents used in the following examples can be obtained from commercial sources unless otherwise specified.

### 1. EXAMPLE 1

### Preparation of 2-(2-(tert-butyl)phenoxy)-N-(4-hydroxyphenyl)acetamide

### 1.1. Preparation of tert-butyl 2-(2-(tert-butyl)phenoxy) acetate

2-(tert-butyl)phenol (0.92 g, 6 mmol) and Cs₂CO₃ (3.9 g, 12 mmol) were dissolved in 6 ml of acetone, then 2-bromoacetate tert-butyl (2.39 g, 12 mmol) was added, and the reaction was carried out overnight at 55° C. After the reaction was complete, the mixture was filtered and the filtrate was concentrated and purified by silica gel chromatography to obtain a white solid tert-butyl 2-(2-(tert-butyl)phenoxy)acetate. ¹H NMR (400 MHz, CDCl₃) δ 7.33 (dd, J=7.7, 1.7 Hz, 1H), 7.18 (ddd, J=8.0, 7.3, 1.7 Hz, 1H), 6.95 (td, J=7.5, 1.2 Hz, 1H), 6.74 (dd, J=8.1, 1.2 Hz, 1H), 4.56 (s, 2H), 1.52 (s, 9H), 1.45 (s, 9H).

### 1.2. Preparation of 2-(2-(tert-butyl)phenoxy)acetic acid

2-(2-(tert-butyl)phenoxy)tert-butyl acetate (0.92 g, 6 mmol) was dissolved in 4 ml of dichloromethane, then 2ml of trifluoroacetic acid was slowly added dropwise. After the reaction was stirred at room temperature for about 2 hours, it was concentrated to obtain 2-(2-(tert-butyl)phenoxy)acetic acid without further purification. ¹H NMR (400 MHz, CDCl₃) δ 7.33 (dd, J=7.8, 1.7 Hz, 1H), 7.22-7.14 (m, 1H), 6.97 (td, J=7.6, 1.2 Hz, 1H), 6.75 (dd, J=8.1, 1.2 Hz, 1H), 4.72 (s, 2H), 1.42 (s, 9H).

### 1.3. Preparation of 2-(2-(tert-butyl)phenoxy)-N-(4-hydroxyphenyl)acetamide

2-(2-(tert-butyl)phenoxy)acetic acid (42 mg, 0.2 mmol) was added to 0.5 ml oxalyl chloride followed by a catalytic amount of DMF. After the reaction was stirred at room temperature for 1-2 hours, it was spin-dried in vacuo. Dry THF (1 ml), 4-aminophenol (26 mg, 0.24 mmol) and Et 3 N (33 μl, 0.24 mmol) were then added to the solid. After stirring for 0.5 hour, the mixture was concentrated in vacuo and purified by silica gel chromatography to obtain a white solid 2-(2-(tert-butyl)phenoxy)-N-(4-hydroxyphenyl)acetamide. ¹H NMR (400 MHz, Acetone-d6) δ 8.86 (s, 1H), 7.57-7.45 (m, 2H), 7.32 (dd, J=7.7, 1.6 Hz, 1H), 7.20 (ddd, J=8.1, 7.2, 1.7 Hz, 1H), 7.05-6.89 (m, 2H), 6.81 (d, J=8.9 Hz, 2H), 4.70 (s, 2H), 1.45 (s, 9H).

### 2. EXAMPLE 2

### Preparation of 2-(2-(tert-butyl)phenoxy)-N-phenylacetamide

Referring to Example 1 (replacing 4-aminophenol in step 1.3 with aniline), Example 2 was obtained as a white solid. ¹H NMR (400 MHz, CDCl₃) δ 8.40 (s, 1H), 7.66-7.58 (m, 2H), 7.40 (td, J=7.3, 1.6 Hz, 3H), 7.28-7.23 (m, 1H), 7.22-7.16 (m, 1H), 7.05 (td, J=7.6, 1.2 Hz, 1H), 6.93 (dd, J=8.2, 1.2 Hz, 1H), 4.71 (s, 2H), 1.54 (s, 9H). ¹³C NMR (101 MHz, CDCl₃) δ 166.43, 155.80, 138.12, 136.99, 129.21, 127.64, 127.30, 124.85, 122.31, 119.69, 113.21, 68.07, 34.77, 30.17.

### 3. EXAMPLE 3

### Preparation of 2-(2-(tert-butyl)phenoxy)-N-(3-hydroxyphenyl)acetamide

Referring to Example 1 (replacing 4-aminophenol in step 1.3 with 3-aminophenol), Example 3 was obtained as a white solid. ¹H NMR (400 MHz, CDCl₃) δ 8.49 (s, 1H), 7.97 (t, J=2.2 Hz, 1H), 7.40 (dd, J=7.9, 1.7 Hz, 1H), 7.35 (s, 1H), 7.24 (q, J=8.2 Hz, 2H), 7.05 (td, J=7.6, 1.2 Hz, 1H), 6.92 (dd, J=8.2, 1.1 Hz, 1H), 6.70 (ddd, J=20.4, 8.0, 2.2 Hz, 2H), 4.73 (s, 2H), 1.52 (s, 9H). ¹³C NMR (101 MHz, CDCl₃) δ 167.29, 157.32, 155.58, 138.09, 137.72, 130.04, 127.67, 127.35, 122.47, 113.29, 112.34, 110.82, 107.16, 67.82, 34.75, 30.18.

### 4. EXAMPLE 4

### Preparation of 2-(2-(tert-butyl)phenoxy)-N-(2-hydroxyphenyl)acetamide

Referring to Example 1 (replacing 2-aminophenol in step 1.3 with 3-aminophenol), Example 4 was obtained as a yellow-brown solid. ¹H NMR (400 MHz, CDCl₃) δ 8.67 (s, 1H), 8.64 (s, 1H), 7.41 (dd, J=7.8, 1.8 Hz, 1H), 7.28-7.24 (m, 1H), 7.22-7.16 (m, 1H), 7.13 (dt, J=8.0, 1.5 Hz, 1H), 7.10-7.03 (m, 2H), 6.93 (td, J=8.0, 1.2 Hz, 2H), 4.78 (s, 2H), 1.52 (s, 9H). ¹³C NMR (101 MHz, CDCl₃) δ 168.22, 155.57, 148.42, 138.24, 127.69, 127.52, 127.43, 124.70, 122.63, 121.98, 120.69, 119.72, 113.27, 67.72, 34.78,

### 5. EXAMPLE 5

### Preparation of 2-(2-(tert-butyl)phenoxy)-N-(4-methoxyphenyl)acetamide

Referring to Example 1 (replacing 4-aminophenol in step 1.3 with 4-methoxyaniline), Example 5 was obtained as a white solid. ¹H NMR (400 MHz, CDCl₃) δ 8.27 (s, 1H), 7.53-7.48 (m, 2H), 7.39 (dd, J=7.8, 1.7 Hz, 1H), 7.25 (ddd, J=8.8, 7.6, 1.7 Hz, 1H), 7.04 (td, J=7.6, 1.2 Hz, 1H), 6.95-6.89 (m, 3H), 4.69 (s, 2H), 3.83 (s, 3H), 1.52 (s, 9H). 13C NMR (101 MHz, CDCl₃) δ 166.22, 156.76, 155.86, 138.12, 130.09, 127.63, 127.28, 122.26, 121.45, 114.31, 113.23, 68.07, 55.53, 34.76, 30.15.

### 6. EXAMPLE 6

### Preparation of ethyl 4-(2-(2-(tert-butyl)phenoxy)acetamido) benzoate

Referring to Example 1 (replacing 4-aminophenol in step 1.3 with ethyl 4-aminobenzoate), Example 6 was obtained as a white solid. ¹H NMR (400 MHz, CDCl₃) δ 8.54 (s, 1H), 8.08 (d, J=8.6 Hz, 2H), 7.68 (d, J=8.7 Hz, 2H), 7.41 (dd, J=7.8, 1.7 Hz, 1H), 7.26 (ddd, J=8.1, 7.4, 1.8 Hz, 1H), 7.06 (td, J=7.6, 1.2 Hz, 1H), 6.91 (dd, J =8.1, 1.3 Hz, 1H), 4.71 (s, 2H), 4.40 (q, J=7.1 Hz, 2H), 1.53 (s, 9H), 1.42 (t, J=7.1 Hz, 3H). ¹³C NMR (101 MHz, CDCl₃) δ 166.71, 165.98, 155.67, 140.91, 138.13, 130.96, 127.68, 127.39, 126.62, 122.49, 118.78, 113.24, 68.09, 60.97, 34.77, 30.19, 14.37.

### 7. EXAMPLE 7

### Preparation of 2-(2-(tert-butyl)phenoxy)-N-(4-(dimethylamino)phenyl)acetamide

Referring to Example 1, Example 7 was obtained as a yellow solid. ¹H NMR (400 MHz, CDCl₃) δ 8.17 (s, 1H), 7.42 (d, J=9.0 Hz, 2H), 7.36 (dd, J=7.8, 1.7 Hz, 1H), 7.22 (td, J=7.8, 1.7 Hz, 1H), 7.00 (td, J=7.5, 1.2 Hz, 1H), 6.89 (dd, J=8.2, 1.2 Hz, 1H), 6.73 (d, J=8.9 Hz, 2H), 4.66 (s, 2H), 2.93 (s, 6H), 1.49 (s, 9H). ¹³C NMR (101 MHz, CDCl₃) δ 166.00, 155.96, 148.31, 138.15, 127.59, 127.21, 126.75, 122.15, 121.47, 113.26, 113.06, 68.14, 40.90, 34.75, 30.14.

### 8. EXAMPLE 8

### Preparation of 2-(2-(tert-butyl)phenoxy)-N-(4-chlorophenyl)acetamide

Referring to Example 1, Example 8 was obtained as a white solid. The yield was 21%. ¹H NMR (400 MHz, CDCl₃) δ 8.39 (s, 1H), 7.60 -7.54 (m, 2H), 7.40 (dd, J=7.8, 1.7 Hz, 1H), 7.38 -7.32 (m, 2H), 7.25 (dd, J=7.9, 1.7 Hz, 1H), 7.06 (td, J=7.6, 1.2 Hz, 1H), 6.91 (dd, J=8.2, 1.2 Hz, 1H), 4.70 (s, 2H), 1.53 (s, 9H). ¹³C NMR (101 MHz, CDCl₃) δ 166.49, 155.74, 138.14, 135.56, 129.85, 129.22, 127.66, 127.35, 122.44, 120.88, 113.26, 68.08, 34.75, 30.19.

### 9. EXAMPLE 9

### Preparation of 2-(2-(tert-butyl)phenoxy)-N-(4-fluorophenyl)acetamide

Referring to Example 1, Example 9 was obtained as a yellow solid. The yield was 40%. ¹H NMR (400 MHz, CDCl₃) δ 8.33 (s, 1H), 7.59-7.48 (m, 2H), 7.37 (dd, J=7.8, 1.7 Hz, 1H), 7.26-7.18 (m, 1H), 7.11-6.99 (m, 3H), 6.89 (dd, J=8.2, 1.2 Hz, 1H), 4.67 (s, 2H), 1.49 (s, 9H). ¹³CNMR (101 MHz, CDCl₃) δ 166.41, 159.65 (d, J=244.32 Hz), 155.78, 138.14, 133.00, 127.65, 127.33, 122.39, 121.44 (d, J=7.95 Hz), 115.87 (d, J=22.65 Hz), 113.25, 68.06, 34.75, 30.17. ¹⁹F NMR (376 MHz, CDCl₃) δ −117.20.

### 10. EXAMPLE 10

### Preparation of 2-(2-(tert-butyl)phenoxy)-N-(p-tolyl)acetamide

Referring to Example 1, Example 10 was obtained as a yellow solid. The yield was 45%. ¹H NMR (400 MHz, CDCl₃) δ 8.29 (s, 1H), 7.48-7.42 (m, 2H), 7.36 (dd, J=7.8, 1.7 Hz, 1H), 7.22 (ddd, J=8.1, 7.4, 1.7 Hz, 1H), 7.18-7.13 (m, 2H), 7.01 (td, J=7.5, 1.2 Hz, 1H), 6.88 (dd, J=8.2, 1.2 Hz, 1H), 4.65 (s, 2H), 2.33 (s, 3H), 1.49 (s, 9H). ¹³C NMR (101 MHz, CDCl₃) δ 166.29, 155.86, 138.14, 134.52, 134.45, 129.67, 127.63, 127.27, 122.26, 119.74, 113.24, 68.10, 34.76, 30.17, 20.93.

### 11. EXAMPLE 11

### Preparation of 2-(2-(tert-butyl)phenoxy)-N-(4-phenoxyphenyl)acetamide

Referring to Example 1, Example 11 was obtained as a yellow solid. The yield was 41%. ¹H NMR (400 MHz, CDCl₃) δ 8.37 (s, 1H), 7.58 (d, J=8.9 Hz, 2H), 7.44-7.32 (m, 3H), 7.26 (dd, J=7.8, 1.7 Hz, 1H), 7.13 (t, J=7.4 Hz, 1H), 7.09-6.99 (m, 5H), 6.93 (dd, J=8.2, 1.2 Hz, 1H), 4.71 (s, 2H), 1.53 (s, 9H). ¹³C NMR (101 MHz, CDCl₃) δ 166.35, 157.44, 155.82, 153.94, 138.13, 132.46, 129.77, 127.65, 127.32, 123.20, 122.34, 121.40, 119.76, 118.51, 113.24, 68.07, 34.77, 30.18.

### 12. EXAMPLE 12

### Preparation of N-([[1,1′-biphenyl]-4-yl]-2-(2-(tert-butyl)phenoxy)acetamide

Referring to Example 1, Example 12 was obtained as a yellow solid. The yield was 41%. ¹H NMR (400 MHz, CDCl₃) δ 8.45 (s, 1H), 7.69 (dd, J=8.7, 2.2 Hz, 2H), 7.66-7.58 (m, 4H), 7.47 (ddd, J=7.9, 6.8, 1.4 Hz, 2H), 7.44-7.34 (m, 2H), 7.29-23 (m, 1H), 7.06 (td, J=7.6, 1.3 Hz, 1H), 6.94 (dd, J=8.2, 1.3 Hz, 1H), 4.73 (s, 2H), 1.55 (s, 9H). ¹³C NMR (101 MHz, CDCl₃) δ 166.46, 155.81, 140.41, 138.16, 137.79, 136.25, 128.83, 127.82, 127.66, 127.32, 127.25, 126.90, 122.36, 120.01, 113.26, 68.12, 34.78, 30.20.

### 13. EXAMPLE 13

### 2-(3-(tert-butyl)phenoxy)-N-(4-hydroxyphenyl)acetamide

Referring to Example 1, Example 13 was obtained as a white solid. The yield was 58%. ¹H NMR (400 MHz, CDCl₃) δ 8.28 (s, 1H), 7.42 (d, J=8.8 Hz, 2H), 7.32 (d, J=8.0 Hz, 1H), 7.12 (ddd, J=7.9, 1.8, 0.9 Hz, 1H), 7.05 (dd, J=2.6, 1.8 Hz, 1H), 6.86-6.78 (m, 3H), 5.83 (s, 1H), 4.65 (s, 2H), 1.35 (s, 9H). ¹³C NMR (101 MHz, CDCl₃) δ 166.78, 156.90, 153.41, 129.43, 122.73, 119.63, 115.87, 112.72, 111.14, 67.56, 34.86, 31.29.

### 14. EXAMPLE 14

### Preparation of 2-(4-(tert-butyl)phenoxy)-N-(4-hydroxyphenyl)acetamide

Referring to Example 1, Example 14 was obtained as a white solid. The yield was 46%. ¹H NMR (400 MHz, CDCl₃) δ 8.26 (d, J=3.9 Hz, 1H), 7.44-7.35 (m, 4H), 6.94 (d, J=8.9 Hz, 2H), 6.83 (d, J=8.8 Hz, 2H), 4.62 (s, 2H), 1.34 (s, 9H). ¹³C NMR (400 MHz, CDCl₃) δ 166.90, 154.79, 153.44, 145.32, 129.31, 126.70, 122.65, 115.87, 114.35, 67.68, 34.22, 31.47.

### 15. EXAMPLE 15

### Preparation of N-(4-hydroxyphenyl)-2-(o-tolyloxy)acetamide

Referring to Example 1, Example 15 was obtained as a white solid. The yield was 52%. ¹H NMR (400 MHz, MeOD-d₄) δ 7.38 (d, J=8.9 Hz, 2H), 7.24-7.11 (m, 2H), 6.97-6.86 (m, 2H), 6.78 (d, J=8.8 Hz, 2H), 4.65 (s, 2H), 2.35 (s, 3H). ¹³C NMR (101 MHz, MeOD-d₄) δ 167.92, 156.09, 154.49, 130.53, 129.17, 126.78, 126.64, 122.42, 121.31, 114.87, 111.49, 67.64, 15.03.

### 16. EXAMPLE 16

### Preparation of N-(4-hydroxyphenyl)-2-phenoxyacetamide

Referring to Example 1, Example 16 was obtained as a white solid. The yield was 55%. ¹H NMR (400 MHz, DMSO-d₆) δ 9.83 (s, 1H), 9.26 (s, 1H), 7.41 (d, J=8.9 Hz, 2H), 7.32 (dd, J=8.8, 7.2 Hz, 2H), 7.03-6.94 (m, 3H), 6.71 (d, J=8.8 Hz, 2H), 4.64 (s, 2H). ¹³C NMR (101 MHz, DMSO-d 6) δ 166.32, 158.28, 154.15, 130.38, 129.96, 122.06, 121.60, 115.50, 115.12, 67.57.

### 17. EXAMPLE 17

### Preparation of 2-([1,1′-biphenyl]-2-yloxy)-N-(4-hydroxyphenyl)acetamide

Referring to Example 1, Example 17 was obtained as a white solid. The yield was 20%. ¹H NMR (400 MHz, MeOD-d₄) δ 7.62-7.57 (m, 2H), 7.48 (t, J=7.5 Hz, 2H), 7.43-7.33 (m, 3H), 7.22-7.08 (m, 4H), 6.77-6.70 (m, 2H), 4.59 (s, 2H). ¹³C NMR (101 MHz, MeOD-d₄) δ 167.00, 154.42, 138.48, 131.38, 130.47, 129.19, 128.85, 128.71, 128.07, 126.95, 122.11, 121.67, 114.85, 113.40, 67.81.

### 18. EXAMPLE 18

### Preparation of 2-(2,6-di-tert-butylphenoxy)-N-(4-hydroxyphenyl)acetamide

Referring to Example 1, Example 18 (76 mg, 38%) was obtained as a yellow solid. ¹H NMR (400 MHz, CDCl₃) δ 8.40 (s, 1H), 7.44-7.38 (m, 2H), 7.28 (d, J=7.9 Hz, 2H), 7.06 (t, J=7.8 Hz, 1H), 6.82 (d, J=8.8 Hz, 2H), 6.42 (d, J=2.1 Hz, 1H), 4.40 (s, 2H), 1.44 (s, 18H). ¹³C NMR (101 MHz, CDCl₃) δ 166.75, 154.74, 153.75, 143.16, 129.20, 127.25, 124.29, 122.58, 115.96, 74.53, 35.85, 32.32.

### 19. EXAMPLE 19

### Preparation of N-(4-hydroxyphenyl)-2-(m-tolyloxy)acetamide

Referring to Example 1, Example 19 (121 mg, 58%) was obtained as a brown solid. ¹H NMR (400 MHz, CDCl₃) δ 8.26 (s, 1H), 7.40 (d, J=8.5 Hz, 2H), 7.25 (t, J=7.9 Hz, 1H), 6.94-6.77 (m, 5H), 6.16 (s, 1H), 4.63 (s, 2H), 2.38 (s, 3H). ¹³C NMR (101 MHz, CDCl₃) δ 166.56, 157.03, 153.19, 140.13, 129.63, 129.55, 123.28, 122.50, 115.82, 115.64, 111.69, 67.50, 21.52.

### 20. EXAMPLE 20

### Preparation of 1-(3-tert-butyl)benzyl-3-(4-hydroxyl)phenylurea

### 20.1. Preparation of 3-tert-butylbenzonitrile

1-Bromo-3-(tert-butyl)benzene (1.00 g, 4.6 mmol) was dissolved in 2 ml DMF, then CuCN (0.48 g, 5.2 mmol) was added. After refluxing for about 2 hours, the reaction mixture was cooled to room temperature. Then 1 ml of diethylamine and 6 ml of water were added. Extracted 3 times with 15 ml Et₂O. The organic layers were combined, washed with saturated NaCl, and dried with anhydrous Na₂SO₄. After filtration, the organic layer was concentrated under vacuum and purified by silica gel chromatography to obtain 3-tert-butylbenzonitrile as a colorless oil. ¹H NMR (400 MHz, CDCl₃) δ 7.67 (s, 1H), 7.63 (d, J=10.2 Hz, 1H), 7.47 (dd, J=7.6, 1.4 Hz, 1H), 7.40 (t, J=7.7 Hz, 1H), 1.33 (s, 9H).

### 20.2. Preparation of (3-(tert-butyl)phenyl)methylamine

LiAlH₄ (0.160 g, 4.2 mmol) was suspended in 3 ml THF, cooled to 0° C., and 3-tert-butylbenzonitrile (0.334 g, 2.1 mmol) was added dropwise with vigorous stirring. After stirring for 2 hours, 0.16 ml of water, 0.32 ml of 15% NaOH and 0.48 ml of water were sequentially added to the reaction. The precipitate was filtered and the organic layer was separated and concentrated to obtain the product without further purification. ¹H NMR (400 MHz, CDCl₃) δ 7.33 (s, 1H), 7.30-7.25 (m, 2H), 7.13 (tt, J=3.2, 1.8 Hz, 1H), 3.86 (s, 2H), 1.33 (s, 9H).

### 20.3 Preparation of 1-(3-tert-butyl)benzyl-3-(4-methoxy)phenylurea

(3-(tert-butyl)phenyl)methanamine (0.245 g, 1.5 mmol) was added to a solution of 4-methoxyphenyl ester (0.27g, 1.8mmol) in MeOH (2m1) at 0° C. After stirring for about 1 hour, the solution was concentrated under reduced pressure and purified by silica gel chromatography to obtain 1-(3-(tert-butyl)benzyl)-3-(4-methoxyphenyeurea as a white solid. ¹H NMR (400 MHz, Acetone-d₆) δ 7.78 (s, 1H), 7.43-7.36 (m, 3H), 7.33-7.20 (m, 2H), 7.15 (d, J=7.3 Hz, 1H), 6.81 (d, J=9.0 Hz, 2H), 6.07 (s, 1H), 4.39 (d, J=5.8 Hz, 2H), 3.73 (s, 3H), 1.30 (s, 9H).

### 20.4. Preparation of 1-(3-tert-butyl)benzyl-3-(4-hydroxyl)phenylurea

Under nitrogen protection, 1-(3-(tert-butyl)benzyl)-3-(4-methoxyphenyl)urea (0.156 g, 0.5 mmol) was dissolved in 2 ml DCM and cooled to −78° C., and then BBr₃ (0.48 ml, 5mmol) was added slowly. After stirring overnight, 2 ml of cold water was slowly added to the reaction mixture. The layers were separated, and the aqueous layer was extracted 3 times with 3 ml EtOAc. The organic layers were combined, washed with saturated NaCl, dried with anhydrous Na₂SO₄, filtered, concentrated and purified by silica gel chromatography to obtain 1-(3-tert-butyl)benzyl-3-(4-hydroxyl)phenylurea. ¹H NMR (400 MHz, Acetone-d₆) δ 8.02 (s, 1H), 7.71 (s, 1H), 7.40 (d, J=1.9 Hz, 1H), 7.33-7.20 (m, 4H), 7.16-7.08 (m, 1H), 6.72 (d, J=8.8 Hz, 2H), 6.07 (t, J=6.1 Hz, 1H), 4.38 (d, J=5.8 Hz, 2H), 1.30 (s, 9H). ¹³C NMR (101 MHz, Acetone-d₆) δ 155.76, 152.56, 150.95, 140.19, 132.52, 128.03, 124.46, 124.25, 123.63, 120.64, 115.11, 43.61, 34.27, 30.78.

### 21. EXAMPLE 21

### Preparation of 2-(2-(tert-butyl)-6-cyanophenoxy)-N-(4-hydroxyphenyl)acetamide

Referring to Example 1, Example 21 was obtained as a brown solid. The yield was 82%. ¹H NMR (400 MHz, Acetone-d6) δ 9.24 (s, 1H), 8.30 (s, 1H), 7.75 (dd, J=8.0, 1.9 Hz, 1H), 7.65 (dd, J=7.6, 1.9 Hz, 1H), 7.62-7.57 (m, 2H), 7.30 (t, J=7.8 Hz, 1H), 6.87-6.80 (m, 2H), 4.87 (s, 2H), 1.47 (s, 9H). ¹³C NMR (101 MHz, Acetone-d6) δ 164.83, 159.36, 154.16, 143.94, 132.68, 132.42, 130.37, 124.70, 121.98, 116.68, 115.11, 106.65, 72.99, 34.96, 30.00.

### 22. EXAMPLE 22

### Preparation of 2-(3-(tert-butyl)phenoxy)-N-(4-hydroxybenzyl)acetamide

Referring to Example 1, Example 22 (139 mg, 65%) was obtained as a yellow solid. ¹H NMR (400 MHz, CDCl₃) δ 7.22 (t, J=8.0 Hz, 1H), 7.15-7.11 (m, 2H), 7.05 (ddd, J=7.8, 1.8, 0.9 Hz, 1H), 7.01-6.97 (m, 1H), 6.93 (t, J=2.2 Hz, 1H), 6.82 (d, J=8.5 Hz, 2H), 6.69 (dd, J=8.2, 2.6 Hz, 1H), 4.55 (s, 2H), 4.47 (d, J=5.9 Hz, 2H), 1.28 (s, 9H). ¹³C NMR (101 MHz, CDCl₃) δ 168.80, 156.96, 155.94, 153.51, 129.31, 129.23, 129.01, 119.39, 115.75, 112.60, 111.01, 67.28, 42.69, 34.81, 31.26.

### 23. EXAMPLE 23

### Preparation of N-(4-hydroxyphenyl)-2-(2-(trifluoromethyl)phenoxy)acetamide

Referring to Example 1, Example 23 (115 mg, 58%) was obtained as a yellow solid. ¹H NMR (400 MHz, Acetone-d6) δ 8.72 (s, 1H), 8.29 (s, 1H), 7.68 (ddd, J=14.2, 7.8, 1.6 Hz, 2H), 7.53-7.44 (m, 2H), 7.32 (d, J=8.3 Hz, 1H), 7.20 (tt, J=7.6, 0.9 Hz, 1H), 6.88-6.72 (m, 2H), 4.80 (s, 2H). ¹³C NMR (101 MHz, Acetone-d6) δ 164.71, 155.53 (q, J=1.8 Hz), 154.13, 134.18, 130.21, 126.94 (q, J=5.2 Hz), 124.10 (q, J=270 Hz), 121.41, 121.09, 118.08(q, J=36 Hz), 115.30, 114.06, 67.91. ¹⁹F NMR (376 MHz, Acetone-d6 δ −62.22.

### 24. EXAMPLE 24

### Preparation of N-(4-hydroxyphenyl)-2-(2-isopropylphenoxy)acetamide

Referring to Example 1, Example 24 (118 mg, 43%) was obtained as a white solid. ¹H NMR (400 MHz, Acetone-d₆) δ 8.87 (s, 1H), 8.25 (s, 1H), 7.55-7.44 (m, 2H), 7.27 (dd, J=7.8, 1.7 Hz, 1H), 7.25-7.10 (m, 1H), 7.04-6.95 (m, 2H), 6.87-6.75 (m, 2H), 4.64 (s, 2H), 3.49 (p, J=6.9 Hz, 1H), 1.26 (d, J=6.9 Hz, 6H). ¹³C NMR (101 MHz, Acetone-d₆) δ 165.92, 155.13, 154.04, 136.98, 130.38, 126.79, 126.05, 121.79, 121.50, 115.16, 112.36, 68.19, 26.56, 22.21.

### 25. EXAMPLE 25

### Preparation of 1-(2-tert-butyl)benzyl-3-(4-hydroxyl)phenylurea

Referring to Example 20, Example 25 (148 mg, 71%) was obtained as a white solid. ¹H NMR (400 MHz, Acetone-d₆) δ 8.15 (s, 1H), 7.82 (s, 1H), 7.48-7.33 (m, 2H), 7.29-7.21 (m, 2H), 7.20-7.08 (m, 2H), 6.76-6.69 (m, 2H), 6.02 (s, 1H), 4.63 (d, J=5.3 Hz, 2H), 1.40 (s, 9H). ¹³C NMR (101 MHz, Acetone-d₆) δ 155.92, 152.70, 147.37, 138.10, 132.25, 130.50, 126.94, 126.18, 125.89, 120.71, 115.24, 42.34, 35.33, 31.19.

### 26. EXAMPLE 26

### Preparation of 2-(2-(tert-butyl)phenoxy)-N-(4-sulfamoylphenyl)acetamide

Referring to Example 1, Example 26 (159 mg, 68%) was obtained as a brown solid. ¹H NMR (400 MHz, DMSO-d₆) δ 10.51 (s, 1H), 7.79 (s, 4H), 7.30-7.24 (m, 3H), 7.18 (td, J=7.7, 1.7 Hz, 1H), 6.97-6.85 (m, 2H), 4.79 (s, 2H), 1.39 (s, 9H). ¹³C NMR (101 MHz, DMSO-d₆) δ 167.39, 157.17, 141.93, 139.12, 137.97, 127.61, 127.27, 126.90, 121.33, 119.26, 112.95, 67.73, 34.98, 30.23.

### 27. EXAMPLE 27

### Preparation of (4-(2-(2-(2-(tert-butyl)phenoxy)acetamido)phenyl)boronic acid

Referring to Example 1, Example 27 (131 mg, 56%) was obtained as a white solid. ¹H NMR (400 MHz, Acetone-d₆) δ 9.10 (s, 1H), 7.87 (d, J=8.5 Hz, 2H), 7.69 (d, J=8.5 Hz, 2H), 7.33 (dd, J=7.8, 1.7 Hz, 1H), 7.20 (ddd, J=8.2, 7.3, 1.7 Hz, 1H), 7.10 (s, 2H), 7.01 (dd, J=8.2, 1.2 Hz, 1H), 6.95 (td, J=7.5, 1.2 Hz, 1H), 4.76 (s, 2H), 1.46 (s, 9H). ¹³C NMR (101 MHz, Acetone-d₆) δ 166.42, 156.86, 140.22, 138.10, 135.02, 127.28, 126.69, 121.52, 118.23, 113.36, 68.21, 34.46, 29.54.

### 28. EXAMPLE 28

### Preparation of 2-(2-(tert-butyl)phenoxy)-N-(4-(hydroxymethyl)phenyl)acetamide

Referring to Example 1, Example 28 (108 mg, 41%) was obtained as a brown solid. ¹H NMR (400 MHz, CDCl₃) δ 8.40 (s, 1H), 7.63-7.57 (m, 2H), 7.40 (td, J=4.9, 2.5 Hz, 3H), 7.27-7.22 (m, 1H), 7.05 (td, J=7.6, 1.2 Hz, 1H), 6.91 (dd, J=8.2, 1.2 Hz, 1H), 4.70 (d, J=2.3 Hz, 4H), 1.53 (s, 9H). ¹³C NMR (101 MHz, CDCl₃) δ 166.49, 155.79, 138.14, 137.49, 136.39, 127.93, 127.63, 127.31, 122.34, 119.80, 113.25, 68.09, 64.88, 34.76, 30.17.

### 29. EXAMPLE 29

### Preparation of tert-butyl (4-(2-(2-(tert-butyl)phenoxy)acetamido)phenyl)carbamate

Referring to Example 1, Example 29 (140 mg, 51%) was obtained as a yellow solid. ¹H NMR (400 MHz, CDCl₃) δ 8.32 (s, 1H), 7.57-7.49 (m, 2H), 7.45-7.32 (m, 3H), 7.27-7.22 (m, 1H), 7.04 (td, J=7.6, 1.2 Hz, 1H), 6.91 (dd, J=8.1, 1.2 Hz, 1H), 6.53 (s, 1H), 4.68 (s, 2H), 1.53 (d, J=10.9 Hz, 18H). ¹³C NMR (101 MHz, CDCl₃) δ 166.27, 155.85, 152.73, 138.15, 135.25, 132.18, 127.63, 127.30, 122.30, 120.49, 119.22, 113.29, 80.62, 68.11, 34.76, 30.16, 28.36.

### Example 30, Preparation of N-(4-aminophenyl)-2-(2-(tert-butyl)phenoxy)acetamide

Example 34 (0.2 g, 0.5 mmol) was dissolved in a solution of 2 ml DCM and 1 ml TFA was slowly added at 0° C. After stirring for about 2 h, it was concentrated and then purified by column chromatography to obtain Example 30 (88 mg, 31%) as a yellow solid. ¹H NMR (400 MHz, CDCl₃) δ 8.16 (s, 1H), 7.35 (td, J=7.7, 7.0, 2.0 Hz, 3H), 7.22 (td, J=7.8, 1.7 Hz, 1H), 7.00 (td, J=7.5, 1.2 Hz, 1H), 6.88 (dd, J=8.1, 1.3 Hz, 1H), 6.71-6.65 (m, 2H), 4.65 (s, 2H), 1.48 (s, 9H). ¹³C NMR (101 MHz, CDCl₃) δ 166.08, 155.93, 143.72, 138.15, 128.33, 127.60, 127.22, 122.19, 121.63, 115.45, 113.26, 68.11, 34.75, 30.14.

### 31. EXAMPLE 31

### Preparation of 2-(2-(tert-butyl)phenoxy)-N-(4-carbamoylphenyl)acetamide

Referring to Example 1, Example 31(124 mg, 46%) was obtained as a yellow solid. ¹H NMR (400 MHz, MeOD-d₄) δ 7.93 (d, J=8.6 Hz, 2H), 7.84 (d, J=8.9 Hz, 2H), 7.34 (dd, J=8.0, 1.7 Hz, 1H), 7.19 (t, J=7.7 Hz, 1H), 6.96 (dd, J=8.0, 6.0 Hz, 2H), 4.79 (s, 2H), 1.46 (s, 9H). ¹³C NMR (101 MHz, MeOD-d₄) δ 168.28, 166.17, 156.93, 143.52, 138.21, 128.81, 126.90, 126.47, 122.84, 121.29, 119.43, 112.79, 67.74, 34.33, 29.14.

### 32. EXAMPLE 32

### Preparation of 2-(2-(tert-butyl)phenoxy)-N-(4-cyanophenyl)acetamide

Referring to Example 1, Example 32 (111 mg, 47%) was obtained as a white solid. ¹H NMR (400 MHz, CDCl₃) δ 8.57 (s, 1H), 7.72 (d, J=8.7 Hz, 2H), 7.64 (d, J=8.6 Hz, 2H), 7.38 (dd, J=7.8, 1.7 Hz, 1H), 7.27-7.21 (m, 1H), 7.04 (td, J=7.6, 1.2 Hz, 1H), 6.87 (dd, J=8.2, 1.2 Hz, 1H), 4.69 (s, 2H), 1.50 (s, 9H). ¹³C NMR (101 MHz, CDCl₃) δ 166.97, 155.59, 140.89, 138.11, 133.48, 127.73, 127.46, 122.64, 119.57, 118.66, 113.27, 107.88, 68.06, 34.77, 30.22.

### 33. EXAMPLE 33

### Preparation of 4-(2-(2-(tert-butyl)phenoxy)acetamido)benzoic acid

Referring to Example 1, Example 33 (120 mg, 43%) was obtained as a brown solid. ¹H NMR (400 MHz, CDCl₃) δ 8.56 (s, 1H), 8.16-8.10 (m, 2H), 7.70 (d, J=8.6 Hz, 2H), 7.38 (dd, J=7.8, 1.7 Hz, 1H), 7.24-7.21 (m, 1H), 7.03 (td, J=7.6, 1.2 Hz, 1H), 6.89 (dd, J=8.2, 1.2 Hz, 1H), 4.71 (s, 2H), 1.51 (s, 9H). ¹³C NMR (101 MHz, CDCl₃) δ 171.22, 166.90, 155.62, 141.74, 138.11, 131.74, 127.70, 127.42, 125.36, 122.53, 118.90, 113.24, 68.06, 34.78, 30.21.

### 34. EXAMPLE 34

### Preparation of 2-(2-(tert-butyl)phenoxy)-N-(4-mercaptophenyl)acetamide

Referring to Example 1, Example 34 (313 mg, 85%) was obtained as a white solid. ¹H NMR (400 MHz, CDCl₃) δ 8.31 (s, 1H), 7.47 (dd, J=9.0, 2.5 Hz, 2H), 7.37 (dd, J=7.8, 1.7 Hz, 1H), 7.28 (d, J=8.6 Hz, 2H), 7.22 (ddd, J=8.9, 7.5, 1.7 Hz, 1H), 7.02 (td, J=7.6, 1.2 Hz, 1H), 6.88 (dd, J=8.2, 1.2 Hz, 1H), 4.66 (s, 2H), 3.45 (s, 1H), 1.49 (s, 9H). ¹³C NMR (101 MHz, CDCl₃) δ 166.40, 155.76, 138.14, 135.14, 130.73, 127.64, 127.32, 126.12, 122.38, 120.38, 113.25, 68.08, 34.74, 30.17.

### 35. EXAMPLE 35

### Preparation of 2-(2-(2-(tert-butyl)phenoxy)acetamido)-5-hydroxybenzoic acid

Referring to Example 1, Example 35 was obtained as a white solid. The yield was 41%. ¹H NMR (400 MHz, Acetone-d₆) δ 11.54 (s, 1H), 8.68 (d, J=9.1 Hz, 1H), 7.57 (d, J=3.0 Hz, 1H), 7.32 (dd, J=7.7, 1.7 Hz, 1H), 7.22-7.10 (m, 2H), 7.00-6.95 (m, 2H), 4.70 (s, 2H), 1.44 (s, 9H). ¹³C NMR (101 MHz, Acetone-d₆) δ 168.46, 167.24, 157.64, 152.61, 138.90, 133.64, 127.28, 126.65, 121.99, 121.91, 121.31, 116.99, 116.83, 114.72, 70.01, 34.49, 29.73.

### 36. EXAMPLE 36

### Preparation of 2-(2-(tert-butyl)phenoxy)-N-(2-chloro-4-hydroxyphenyl)acetamide

Referring to Example 1, Example 36 was obtained as a white solid. The yield was 43%. ¹H NMR (400 MHz, DMSO-d₆) δ 9.88 (s, 1H), 9.34 (s, 1H), 7.57 (d, J=8.7 Hz, 1H), 7.26 (d, J=7.7 Hz, 1H), 7.20 (t, J=7.8 Hz, 1H), 7.02-6.85 (m, 3H), 6.77 (dd, J=8.8, 2.8 Hz, 1H), 4.76 (s, 2H), 1.39 (s, 9H). ¹³C NMR (101 MHz, DMSO-d₆) δ 167.12, 157.05, 156.06, 137.99, 127.67, 127.48, 126.91, 125.94, 121.57, 116.09, 115.00, 113.51, 67.97, 34.95, 30.30.

### 37. EXAMPLE 37

### Preparation of 2-(2-(tert-butyl)phenoxy)-N-(3,4-dihydroxyphenyl)acetamide

Referring to Example 1, Example 37 was obtained as a white solid. The yield was 29%. ¹H NMR (400 MHz, Acetone-d₆) δ 8.75 (s, 1H), 7.37 (d, J=2.5 Hz, 1H), 7.32 (dd, J=7.8, 1.7 Hz, 1H), 7.19 (ddd, J=8.8, 7.4, 1.7 Hz, 1H), 7.02-6.89 (m, 3H), 6.77 (d, J=8.5 Hz, 1H), 4.68 (s, 2H), 1.46 (s, 9H). ¹³C NMR (101 MHz, Acetone-d₆) δ 165.66, 156.85, 144.95, 141.69, 138.04, 130.99, 127.29, 126.67, 121.49, 115.08, 113.40, 111.02, 107.74, 68.23, 34.44, 29.54.

### 38. EXAMPLE 38

### Preparation of 2-(2-(tert-butyl)phenoxy)-N-(4-hydroxy-2-(trifluoromethyl)phenyl)acetamide

Referring to Example 1, Example 38 was obtained as a white solid. The yield was 25%. ¹H NMR (400 MHz, CDCl₃) δ 8.35 (s, 1H), 7.50 (d, J=8.7 Hz, 1H), 7.37 (dd, J =7.8, 1.7 Hz, 1H), 7.25-7.21 (m, 1H), 7.12 (s, 1H), 7.03 (td, J=7.6, 1.2 Hz, 1H), 6.98 (d, J=2.8 Hz, 1H), 6.91 (dd, J=8.2, 1.3 Hz, 1H), 6.82 (dd, J=8.8, 2.8 Hz, 1H), 4.76 (s, 2H), 1.44 (s, 9H). ¹³C NMR (101 MHz, CDCl₃) δ 169.25, 156.04, 154.97 (q, J=9.5 Hz), 138.80, 129.27, 127.51, 127.31, 124.99(q, J=30.5 Hz), 123.56(q, J=271 Hz), 122.60, 119.81, 113.80 (q, J=5.2 Hz), 113.71, 68.50, 34.76, 30.03. ¹⁹F NMR (376 MHz, CDCl₃) δ -61.35.

### 39. EXAMPLE 39

### Preparation of 2-(2-(tert-butyl)phenoxy)-N-(4-hydroxyl-3-(trifluoromethyl)phenyl)acetamide

Referring to Example 1, Example 39 was obtained as a white solid. The yield was 19%. ¹H NMR (400 MHz, CDCl₃) δ 9.45 (s, 1H), 8.08 (d, J=2.6 Hz, 1H), 7.88 (dd, J =8.8, 2.6 Hz, 1H), 7.38-7.30 (m, 2H), 7.27-7.16 (m, 1H), 6.98 (t, J=7.6 Hz, 1H), 6.82 (d, J=8.1 Hz, 1H), 4.96 (s, 2H), 1.43 (s, 9H). ¹³C NMR (101 MHz, CDCl₃) δ 167.06, 157.22, 156.25, 138.78, 134.22, 127.18, 127.07, 125.35, 124.12, 124.03 (q, J=233.1 Hz), 123.80 (q, J=27.4 Hz), 121.76, 118.49 (q, J=4.7 Hz), 111.82, 64.88, 34.90, 29.78. ¹⁹F NMR (376 MHz, CDCl₃) δ -61.85.

### 40. EXAMPLE 40

### Preparation of 2-(2-(tert-butyl)phenoxy)-N-(2-fluoro-4-hydroxyphenyl)acetamide

Referring to Example 1, Example 40 was obtained as a white solid. The yield was 48%. ¹H NMR (400 MHz, CDCl₃) δ 8.50 (s, 1H), 8.03 (t, J=9.0 Hz, 1H), 7.36 (dd, J=7.8, 1.7 Hz, 1H), 7.28-7.17 (m, 1H), 7.12 (s, 1H), 7.01 (t, J=7.5 Hz, 1H), 6.88 (d, J=8.2 Hz, 1H), 6.66 (dt, J=10.9, 3.1 Hz, 2H), 4.70 (s, 2H), 1.47 (s, 9H). ¹³C NMR (101 MHz, CDCl₃) δ 167.30, 155.67, 154.40, 153.96(d, J=244 Hz), 138.34, 127.53, 127.32, 123.43, 122.36, 117.41(d, J=11.2 Hz), 113.11, 111.36(d, J=3 Hz), 103.39(d, J=22 Hz), 67.78, 34.74, 30.04. ¹⁹F NMR (376 MHz, CDCl₃) δ -126.47.

### 41. EXAMPLE 41

### Preparation of 2-(2-(tert-butyl)phenoxy)-N-(3-fluoro-4-hydroxyphenyl)acetamide

Referring to Example 1, Example 41 was obtained as a white solid. The yield was 6%. ¹H NMR (400 MHz, CDCl₃) δ 8.28 (s, 1H), 7.62 (dd, J=12.0, 2.2 Hz, 1H), 7.37 (dd, J=7.8, 1.8 Hz, 1H), 7.28-7.19 (m, 1H), 7.07-6.94 (m, 3H), 6.88 (d, J=8.1 Hz, 1H), 5.62 (s, 1H), 4.67 (s, 2H), 1.49 (s, 9H). ¹³C NMR (101 MHz, CDCl₃) δ 166.58, 155.75, 150.65 (d, J=237.9 Hz), 140.84 (d, J=14.4 Hz), 138.15, 129.88 (d, J=9.3 Hz), 127.66, 127.33, 122.42, 117.41 (d, J=2.9 Hz),116.22 (d, J=3.6 Hz), 113.27, 108.60 (d, J=23.1 Hz), 67.99, 34.74, 30.17. ¹⁹F NMR (376 MHz, CDCl₃) δ -137.52.

### 42. EXAMPLE 42

### Preparation of 2-(2-(tert-butyl)phenoxy)-N-(3-chloro-4-hydroxyphenyl)acetamide

Referring to Example 1, the preparation of white solid Example 42 was obtained. The yield was 27%. ¹H NMR (400 MHz, CDCl₃) δ 8.27 (s, 1H), 7.73 (d, J=2.6 Hz, 1H), 7.36 (dd, J=7.8, 1.7 Hz, 1H), 7.25-7.18 (m, 2H), 7.06-6.93 (m, 2H), 6.86 (dd, J=8.2, 1.2 Hz, 1H), 6.15 (s, 1H), 4.66 (s, 2H), 1.48 (s, 9H). ¹³C NMR (101 MHz, CDCl₃) δ 166.76, 155.80, 149.02, 138.20, 130.15, 127.67, 127.35, 122.45, 121.29, 120.38, 120.24, 116.55, 113.36, 68.07, 34.76, 30.20.

43. EXAMPLE 43

### Preparation of 2-(2-(tert-butyl)phenoxy)-N-(3-chloro-4-hydroxyphenyl)acetamide

Referring to Example 1, Example 43 was obtained as a white solid. The yield was 32%. ¹H NMR (400 MHz, DMSO-d₆) δ 9.29 (s, 1H), 9.21 (s, 1H), 7.26 (d, J=7.7 Hz, 1H), 7.22-7.14 (m, 2H), 7.01-6.89 (m, 2H), 6.63 (d, J=2.6 Hz, 1H), 6.58 (dd, J=8.6, 2.7 Hz, 1H), 4.70 (s, 2H), 2.11 (s, 3H), 1.39 (s, 9H). ¹³C NMR (101 MHz, DMSO-d₆) δ 166.90, 157.29, 155.57, 138.02, 134.12, 127.60, 127.36, 127.09, 126.88, 121.42, 117.12, 113.37, 113.13, 68.20, 34.94, 30.27, 18.33.

### 44. EXAMPLE 44

### Preparation of 2-(2-(tert-butyl)phenoxy)-N-(4-hydroxyl-3-methoxyphenyl)acetamide

Referring to Example 1, Example 44 was obtained as a white solid. The yield was 38%. ¹H NMR (400 MHz, CDCl₃) δ 8.28 (s, 1H), 7.55 (d, J=2.4 Hz, 1H), 7.36 (dd, J =7.8, 1.7 Hz, 1H), 7.22 (td, J=9.0, 7.8, 2.9 Hz, 1H), 7.01 (t, J=7.5 Hz, 1H), 6.88 (dd, J=8.4, 6.6 Hz, 2H), 6.71 (dd, J=8.4, 2.4 Hz, 1H), 4.66 (s, 2H), 3.90 (s, 3H), 1.49 (s, 9H). ¹³C NMR (101 MHz, CDCl₃) δ 166.45, 155.87, 146.62, 142.94, 138.16, 129.78, 127.64, 127.30, 122.33, 114.35, 113.34, 112.51, 104.28, 68.11, 56.08, 34.76, 30.18.

### 45. Example 45

### Preparation of 5-(2-(2-(tert-butyl)phenoxy)acetamido)-2-hydroxybenzoic acid methyl ester

Referring to Example 1, Example 45 was obtained as a white solid. The yield was 70%. ¹H NMR (400 MHz, CDCl₃) δ 10.70 (s, 1H), 8.26 (s, 1H), 8.18 (t, J=2.6 Hz, 1H), 7.54 (dt, J=8.9, 2.6 Hz, 1H), 7.40 (dt, J=7.8, 2.1 Hz, 1H), 7.26 (t, J=8.3 Hz, 1H), 7.09-6.98 (m, 2H), 6.91 (dt, J=8.2, 1.7 Hz, 1H), 4.70 (d, J=2.1 Hz, 2H), 3.99 (d, J=2.3 Hz, 3H), 1.52 (d, J=2.1 Hz, 9H). ¹³C NMR (101 MHz, CDCl₃) δ 170.13, 166.55, 158.83, 155.91, 138.25, 128.54, 128.25, 127.65, 127.33, 122.42, 121.30, 118.22, 113.41, 112.28, 68.23, 52.52, 34.76, 30.18.

### 46. EXAMPLE 46

### Preparation of N-(3-acetyl-4-hydroxyphenyl)-2-(2-(tert-butyl)phenoxy)acetamide

Referring to Example 1, Example 46 was obtained as a white solid. The yield was 56%. ¹H NMR (400 MHz, CDCl₃) δ 12.16 (s, 1H), 8.33 (d, J=2.7 Hz, 1H), 8.28 (s, 1H), 7.37 (ddd, J=11.6, 8.4, 2.2 Hz, 2H), 7.23 (dd, J=7.8, 1.7 Hz, 1H), 7.04 (td, J=7.6, 1.2 Hz, 1H), 6.98 (d, J=8.9 Hz, 1H), 6.90 (dd, J=8.2, 1.2 Hz, 1H), 4.69 (s, 2H), 2.67 (s, 3H), 1.50 (s, 9H). ¹³C NMR (101 MHz, CDCl₃) δ 204.46, 166.64, 159.52, 155.81, 138.16, 128.60, 128.33, 127.68, 127.39, 122.48, 121.97, 119.28, 118.99, 113.35, 68.13, 34.77, 30.20, 26.87.

### 47. EXAMPLE 47

### Preparation of 2-(2-(tert-butyl)phenoxy)-N-(4-hydroxyl-3-(hydroxymethyl)phenyl)acetamide

Referring to Example 1, Example 47 was obtained as a white solid. The yield was 37%. ¹H NMR (400 MHz, Acetone-d₆) δ 8.85 (s, 1H), 8.35 (s, 1H), 7.56 (d, J=2.6 Hz, 1H), 7.44 (dd, J=8.6, 2.7 Hz, 1H), 7.32 (dd, J=7.8, 1.7 Hz, 1H), 7.24-7.15 (m, 1H), 7.00 (dd, J=8.2, 1.2 Hz, 1H), 6.95 (td, J=7.6, 1.3 Hz, 1H), 6.78 (d, J=8.5 Hz, 1H), 4.72 (d, J=5.2 Hz, 2H), 4.70 (s, 2H), 4.46 (t, J=5.5 Hz, 1H), 1.45 (s, 9H). ¹³C NMR (101 MHz, Acetone-d₆) δ 165.82, 156.94, 151.62, 138.06, 130.43, 127.94, 127.29, 126.68, 121.48, 119.56, 119.36, 115.05, 113.41, 68.26, 60.55, 34.46, 29.55.

### 48. EXAMPLE 48

### Preparation of 2-(2-(tert-butyl)phenoxy)-N-(2,4-dihydroxyphenyl)acetamide

Referring to Example 1, Example 48 was obtained as a white solid. The yield was 19%. ¹H NMR (400 MHz, Acetone-d₆) δ 9.06 (s, 1H), 8.87 (s, 1H), 8.17 (s, 1H), 7.89 (d, J=8.7 Hz, 1H), 7.34 (dd, J=7.7, 1.8 Hz, 1H), 7.21 (ddd, J=8.8, 7.3, 1.7 Hz, 1H), 7.04 (dd, J=8.3, 1.3 Hz, 1H), 6.97 (td, J=7.5, 1.3 Hz, 1H), 6.48 (t, J=3.1 Hz, 1H), 6.36 (dd, J=8.7, 2.7 Hz, 1H), 4.74 (s, 2H), 1.46 (s, 9H). ¹³C NMR (101 MHz, Acetone-d₆) δ 166.01, 156.50, 154.85, 147.96, 138.09, 127.35, 126.74, 121.69, 121.43, 118.76, 113.44, 106.31, 103.09, 67.94, 34.44, 29.62.

### 49. EXAMPLE 49

### Preparation of 2-(2-(tert-butyl)phenoxy)-N-(5-hydroxypyridin-2-yl)acetamide

Referring to Example 1, Example 49 was obtained as a white solid. The yield was 13%. ¹H NMR (400 MHz, Acetone-d₆) δ 8.98 (s, 1H), 8.67 (s, 1H), 8.12 (d, J=8.9 Hz, 1H), 7.91 (d, J=3.0 Hz, 1H), 7.34 (dd, J=7.8, 1.7 Hz, 1H), 7.30 (dd, J=8.9, 3.0 Hz, 1H), 7.21 (ddd, J=8.2, 7.3, 1.7 Hz, 1H), 7.02 (dd, J=8.2, 1.2 Hz, 1H), 6.96 (td, J=7.5, 1.2 Hz, 1H), 4.80 (s, 2H), 1.48 (s, 9H). ¹³C NMR (101 MHz, Acetone-d₆) δ 166.03, 156.49, 150.75, 144.00, 137.97, 135.35, 127.32, 126.75, 124.43, 121.56, 113.96, 113.02, 67.62, 34.43, 29.54.

### 50. EXAMPLE 50

### Preparation of 2-(2-(tert-butyl)phenoxy)-N-(4-hydroxyl-2-(hydroxymethyl)phenyl)acetamide

Referring to Example 1, Example 50 was obtained as a white solid. The yield was 57%. ¹H NMR (400 MHz, Acetone-d₆) δ 9.29 (s, 1H), 8.27 (s, 1H), 7.80 (d, J=8.5 Hz, 1H), 7.32 (dd, J=7.8, 1.7 Hz, 1H), 7.20 (ddd, J=8.8, 7.3, 1.7 Hz, 1H), 7.01 (dd, J=8.2, 1.3 Hz, 1H), 6.96 (td, J=7.5, 1.2 Hz, 1H), 6.80-6.73 (m, 2H), 4.70 (s, 2H), 4.51 (d, J=5.4 Hz, 2H), 4.43 (t, J=5.4 Hz, 1H), 1.44 (s, 9H). ¹³C NMR (101 MHz, Acetone-d₆) δ 166.71, 157.24, 154.28, 138.59, 134.04, 128.40, 127.27, 126.71, 124.37, 121.80, 114.93, 114.13, 113.99, 69.15, 62.39, 34.49, 29.65.

### 51. EXAMPLE 51

### Preparation of 2-(2-(tert-butyl)-4-methoxyphenoxy)-N-(4-hydroxyphenyeacetamide

Referring to Example 1, Example 51 was obtained as a white solid. The yield was 37%. ¹H NMR (400 MHz, Acetone-d₆) δ 8.80 (s, 1H), 8.22 (s, 1H), 7.54-7.48 (m, 2H), 6.95 (d, J=8.8 Hz, 1H), 6.87 (d, J=3.1 Hz, 1H), 6.84-6.78 (m, 2H), 6.75 (dd, J=8.8, 3.1 Hz, 1H), 4.61 (s, 2H), 3.75 (s, 3H), 1.44 (s, 9H). ¹³C NMR (101 MHz, Acetone-d₆) δ 166.02, 154.39, 153.98, 151.02, 139.72, 130.48, 121.22, 115.20, 114.77, 113.96, 110.28, 69.20, 54.79, 34.52, 29.48.

### 52. EXAMPLE 52

### Preparation of 2-(2-(tert-butyl)-4-hydroxyphenoxy)-N-(4-hydroxyphenyeacetamide

Referring to Example 1 and Example 24, Example 52 was obtained as a white solid. The yield was 37%. ¹H NMR (400 MHz, Acetone-d₆) δ 8.81 (s, 1H), 8.25 (s, 1H), 7.92 (s, 1H), 7.53 (d, J=8.9 Hz, 2H), 6.95-6.75 (m, 4H), 6.66 (dd, J=8.7, 3.0 Hz, 1H), 4.59 (s, 2H), 1.43 (s, 9H). ¹³C NMR (101 MHz, Acetone-d₆) δ 166.22, 153.98, 151.90, 150.18, 139.67, 130.46, 121.25, 115.35, 115.20, 114.15, 112.74, 69.42, 34.38, 29.55.

### 53. EXAMPLE 53

### Preparation of 2-(2-(tert-butyl)-6-methylphenoxy)-N-(4-hydroxyphenyl)acetamide

Referring to Example 1, Example 53 was obtained as a white solid. The yield was 45%. ¹H NMR (400 MHz, CDCl₃) δ 8.49 (s, 1H), 7.45 (d, J=8.8 Hz, 2H), 7.22 (dd, J =7.8, 1.9 Hz, 1H), 7.09 (dd, J=7.5, 1.8 Hz, 1H), 7.03 (t, J=7.6 Hz, 1H), 6.84 (d, J=8.9 Hz, 2H), 6.04 (s, 1H), 4.49 (s, 2H), 2.32 (s, 3H), 1.42 (s, 9H). ¹³C NMR (101 MHz, CDCl₃) δ 167.01, 154.75, 153.45, 142.44, 130.85, 130.34, 129.47, 125.45, 124.59, 122.33, 115.92, 70.96, 35.04, 31.29, 17.16.

### 54. EXAMPLE 54

### Preparation of 2-(2-(tert-butyl)-4-ethylphenoxy)-N-(4-hydroxyphenyl)acetamide

Referring to Example 1, Example 54 was obtained as a white solid. The yield was 52%. ¹H NMR (400 MHz, CDCl₃) δ 8.28 (s, 1H), 7.45-7.36 (m, 2H), 7.17 (d, J=2.2 Hz, 1H), 7.04 (dd, J=8.3, 2.2 Hz, 1H), 6.81 (dd, J=8.6, 7.1 Hz, 3H), 5.71 (s, 1H), 4.64 (s, 2H), 2.61 (q, J=7.6 Hz, 2H), 1.47 (s, 9H), 1.23 (t, J=7.6 Hz, 3H). ¹³C NMR (101 MHz, CDCl₃) 8166.88, 153.91, 153.22, 137.94, 129.69, 126.99, 126.42, 121.99, 115.88, 113.35, 68.26, 34.70, 30.22, 28.33, 15.79.

### 55. EXAMPLE 55

### Preparation of 2-(2-(tert-butyl)-5-methylphenoxy)-N-(4-hydroxyphenyl)acetamide

Referring to Example 1, Example 55 was obtained as a white solid. The yield was 65%. ¹H NMR (400 MHz, CDCl₃) δ 8.28 (s, 1H), 7.44-7.36 (m, 2H), 7.23 (d, J=7.9 Hz, 1H), 6.87-6.79 (m, 3H), 6.70 (d, J=1.7 Hz, 1H), 5.83 (s, 1H), 4.66 (s, 2H), 2.32 (s, 3H), 1.46 (s, 9H). ¹³C NMR (101 MHz, CDCl₃) δ 166.98, 155.67, 153.54, 137.62, 135.17, 129.42, 127.12, 122.87, 122.09, 115.96, 114.25, 67.99, 34.42, 30.26, 21.03.

### 56. EXAMPLE 56

### Preparation of 2-(2-(tert-butyl)-4-fluorophenoxy)-N-(4-hydroxyphenyl)acetamide

Referring to Example 1, Example 56 was obtained as a white solid. The yield was 69%. ¹H NMR (400 MHz, Acetone-d₆) δ 8.91 (s, 1H), 8.26 (s, 1H), 7.50 (d, J=8.9 Hz, 2H), 7.03 (ddd, J=17.2, 9.9, 4.0 Hz, 2H), 6.94 (ddd, J=9.0, 7.5, 3.1 Hz, 1H), 6.81 (d, J=8.8 Hz, 2H), 4.69 (s, 2H), 1.44 (s, 9H). ¹³C NMR (101 MHz, Acetone-d₆) δ 165.77, 157.42 (d, J=236.9 Hz), 154.06, 153.25 (d, J=2.1 Hz), 140.68 (d, J=6.1 Hz), 130.40, 121.37, 115.25, 114.83 (d, J=8.5 Hz), 113.74 (d, J=24.3 Hz),112.70 (d, J=22.7 Hz), 68.98, 34.67, 29.26. ¹⁹F NMR (376 MHz, Acetone-d₆) δ -123.49.

### 57. EXAMPLE 57

### Preparation of 2-(2-(tert-butyl)-4-chlorophenoxy)-N-(4-hydroxyphenyl)acetamide

Referring to Example 1, Example 57 was obtained as a white solid. The yield was 80%. ¹H NMR (400 MHz, CDCl₃) δ 8.18 (s, 1H), 7.37 (d, J=8.8 Hz, 2H), 7.31 (d, J=2.6 Hz, 1H), 7.18 (dd, J=8.7, 2.6 Hz, 1H), 6.85-6.78 (m, 3H), 5.97 (s, 1H), 4.64 (s, 2H), 1.46 (s, 9H). ¹³C NMR (101 MHz, CDCl₃) δ 166.27, 154.39, 153.44, 140.12, 129.37, 127.68, 127.43, 127.15, 122.09, 115.94, 114.44, 68.27, 34.95, 29.92.

### 58. EXAMPLE 58

### Preparation of 2-(2-(tert-butyl)-6-(hydroxymethyl)phenoxy)-N-(4-hydroxyphenyl)acetamide

Referring to Example 1, Example 58 was obtained as a white solid. The yield was 7%. ¹H NMR (400 MHz, Acetone-d₆) δ 9.27 (s, 1H), 8.24 (s, 1H), 7.60 (d, J=8.9 Hz, 1H), 7.35 (ddd, J=15.0, 7.7, 1.8 Hz, 2H), 7.10 (t, J=7.7 Hz, 1H), 6.82 (d, J=8.9 Hz, 1H), 4.73 (d, J=5.3 Hz, 2H), 4.59 (s, 2H), 4.44 (t, J=5.5 Hz, 1H), 1.42 (s, 8H). ¹³C NMR (101 MHz, Acetone-d₆) 8165.99, 155.35, 154.00, 142.39, 135.40, 130.58, 128.78, 126.65, 124.22, 121.60, 115.12, 73.53, 59.77, 34.81, 30.81.

### 59. EXAMPLE 59

### Preparation of 2-(2-bromo-6-(tert-butyl)phenoxy)-N-(4-hydroxyphenyl)acetamide

Referring to Example 1, Example 59 was obtained as a white solid. The yield was 94%. ¹H NMR (400 MHz, Acetone-d6) δ 9.07 (s, 1H), 8.25 (s, 1H), 7.60 (d, J=8.5 Hz, 2H), 7.53 (d, J=7.7 Hz, 1H), 7.43 (d, J=8.0 Hz, 1H), 7.07 (t, J=8.0 Hz, 1H), 6.82 (d, J=8.5 Hz, 2H), 4.63 (s, 2H), 1.44 (s, 9H). ¹³C NMR (101 MHz, Acetone-d6) δ 165.35, 154.10, 153.68, 145.98, 132.31, 130.41, 127.21, 125.79, 121.83, 117.46, 115.03, 101.70, 69.77, 34.33, 31.54.

### 60. Example 60

### Preparation of 2-(2,4-dibromo-6-(tert-butyl)phenoxy)-N-(4-hydroxyphenyl)acetamide

Referring to Example 1, Example 60 was obtained as a white solid. The yield was 99%. ¹H NMR (400 MHz, Acetone-d6) δ 9.15 (s, 1H), 8.32 (s, 1H), 7.71 (d, J=2.5 Hz, 1H), 7.60 (d, J=9.1 Hz, 2H), 7.53 (d, J=2.3 Hz, 1H), 6.84 (d, J=8.7 Hz, 2H), 4.67 (s, 2H), 1.45 (s, 9H). ¹³C NMR (101 MHz, Acetone-d6) δ 162.31, 154.17, 153.41, 148.57, 134.18, 130.28, 130.24, 121.97, 119.02, 117.25, 115.14, 71.79, 37.10, 31.08.

### 61. EXAMPLE 61

### Preparation of 2-(4-bromo-2-(tert-butyl)phenoxy)-N-(4-hydroxyphenyl)acetamide

Referring to Example 1, Example 61 was obtained as a white solid. The yield was 44%. ¹H NMR (400 MHz, Acetone-d6) δ 8.93 (s, 1H), 8.28 (s, 1H), 7.51 (d, J=8.6 Hz, 2H), 7.42 (d, J=2.5 Hz, 1H), 7.36 (dd, J=8.6, 2.4 Hz, 1H), 6.99 (d, J=8.7 Hz, 1H), 6.82 (d, J=8.5 Hz, 2H), 4.74 (s, 2H), 1.46 (s, 9H). ¹³C NMR (101 MHz, Acetone-d6) δ 164.11, 156.35, 153.42, 140.83, 130.46, 129.86, 128.80, 119.69, 116.31, 115.24, 112.62, 67.67, 34.72, 29.21.

### 62. EXAMPLE 62

### Preparation of 4-(tert-butyl)-3-(24(4-hydroxyphenyl)amino)-2-oxoethoxy)methyl benzoate

Referring to Example 1, Example 62 was obtained as a white solid. The yield was 95%. ¹H NMR (400 MHz, Acetone-d6) δ 8.99 (s, 1H), 8.25 (s, 1H), 7.65-7.57 (m, 2H), 7.51 (d, J=7.6 Hz, 2H), 7.45 (dd, J=8.0, 1.6 Hz, 1H), 6.81 (d, J=7.0 Hz, 1H), 4.81 (s, 2H), 3.85 (s, 3H), 1.47 (s, 9H). ¹³C NMR (101 MHz, Acetone-d6) δ 166.05, 164.78, 157.44, 154.02, 143.68, 132.42, 129.37, 127.84, 123.31, 121.29, 116.42, 112.71, 68.82, 49.61, 34.95, 29.21.

### 63. EXAMPLE 63

### Preparation of 4-(tert-butyl)-3-(24(4-hydroxyphenyl)amino)-2-oxoethoxy)benzamide

Referring to Example 1, Example 63 was obtained as a white solid. The yield was 38%. ¹H NMR (400 MHz, MeOD) δ 7.52-7.38 (m, 5H), 6.79 (d, J=8.7 Hz, 2H), 4.77 (s, 2H), 1.49 (s, 9H). ¹³C NMR (101 MHz, MeOD) δ 170.51, 167.06, 157.00, 154.42, 142.56, 132.58, 129.38, 126.65, 122.06, 120.38, 114.97, 112.14, 67.87, 34.63, 28.89.

### 64. EXAMPLE 64

### Preparation of 4-(tert-butyl)-3-(24(4-hydroxyphenyl)amino)-2-oxoethoxy)benzoic acid

Referring to Example 1, Example 64 was obtained as a white solid. The yield was 40%. ¹H NMR (400 MHz, Acetone-d6) δ 9.01 (s, 1H), 7.67-7.59 (m, 2H), 7.51 (d, J=8.6 Hz, 2H), 7.45 (d, J=8.0 Hz, 1H), 6.81 (d, J=8.5 Hz, 2H), 4.82 (s, 2H), 1.48 (s, 9H). ¹³C NMR (101 MHz, Acetone-d6) δ 166.47, 165.42, 156.99, 154.01, 143.53, 130.55, 129.74, 126.89, 122.84, 121.28, 115.22, 113.78, 68.11, 34.93, 29.23.

### 65. EXAMPLE 65

### Preparation of 3-(tert-butyl)-2-(2-((4-hydroxyphenyl)amino)-2-oxoethoxy)methyl benzoate

Referring to Example 1, Example 65 was obtained as a white solid. The yield was 89%. ¹H NMR (400 MHz, Acetone-d6) δ 9.04 (s, 1H), 8.33 (s, 1H), 7.73 (dd, J=7.7, 1.6 Hz, 1H), 7.63 (t, J=8.4 Hz, 3H), 7.21 (t, J=7.8 Hz, 1H), 6.86 (d, J=8.4 Hz, 2H), 4.48 (s, 2H), 3.85 (s, 3H), 1.46 (s, 9H). ¹³C NMR (101 MHz, Acetone-d6) δ 166.66, 165.59, 157.11, 154.07, 143.74, 131.59, 130.48, 130.07, 124.77, 123.90, 121.52, 115.23, 74.33, 51.92, 35.09, 30.54.

### 66. PREPARATION OF EXAMPLE 66

### 2-(2-(tert-butyl)-4-cyanophenoxy)-N-(4-hydroxyphenyl)acetamide

Referring to Example 1, Example 66 was obtained as a white solid. The yield was 93%. ¹H NMR (400 MHz, Acetone-d6) δ 9.10 (s, 1H), 8.37 (s, 1H), 7.64 (d, J=8.8 Hz, 2H), 7.49 (d, J=8.8 Hz, 2H), 7.17 (d, J=8.3 Hz, 1H), 6.82 (d, J=8.8 Hz, 2H), 4.89 (s, 2H), 1.48 (s, 9H). ¹³C NMR (101 MHz, Acetone-d6) δ 164.95, 160.47, 154.08, 139.54, 132.05, 130.66, 130.44, 121.29, 119.05, 115.26, 113.61, 104.33, 67.75, 34.83, 29.00.

### BIOLOGICAL EXAMPLES

1. High-Throughput Screening of NAMPT Agonists

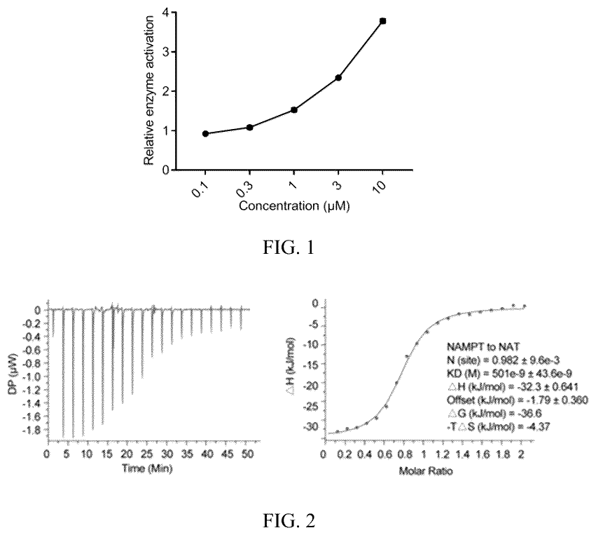

We completed a high-throughput screening aimed at screening for small-molecule agonists of NAMPT. The NAMPT enzyme activity was assayed by a method coupled with the three enzymes: NAMPT, NMNAT1 and alcohol dehydrogenase (ADH). NAMPT synthesizes NMN from NAM and PRPP, NMNAT1 synthesizes NAD from NMN produced in the first step, and ADH converts NAD into detectable fluorescent NADH. In the NAMPT enzyme activity assay, 50 mM Tris-HC1 (pH 28.0), 12 mM magnesium chloride, 1.5% ethanol, 15 μM PRPP, 2.5 mM ATP, 10 mM semicarbazide, 0.2% bovine serum albumin (BSA), 2.4 μg/ml NMNAT, 60 units of ADH and 1 μM NAMPT were used. About 50,000 synthetic small molecules were tested for NAMPT enzyme activity with this system in a 384-well plate, and finally we found 3 compounds exhibited activity in the NAMPT enzyme activity assay. Among these compounds, the compound NAT (the product of Example 1) exhibited the most stable and reproducible NAMPT activation activity (as shown in FIG. 1).

2. Direct Binding of NAT to NAMPT

We verified the direct binding between in vitro expressed and purified NAMPT and NAT by isothermal calorimetric titration (ITC). We performed reverse titration with Microcal PEAQ-ITC (Malvern): 200 μM NAMPT was placed in the titration needle, and 25 μM NAT was placed in the titration cell. The final data were fitted using a single-point model. NAT was bound to NAMPT at a ratio of 1:1, and the equilibrium dissociation constant (Kd) of the binding was about 501 nM (as shown in FIG. 2).

3. Cytoprotective Activity Test of NAT and Derivatives Thereof

Compound 1-66 (wherein Compound 1 was NAT) was synthesized according to preparation Example 1-66 of the present invention. These compounds were evaluated in two separate assays: an in vitro assay of NAMPT enzyme activity and an assay for protection against cell death induced by the NAMPT inhibitor FK866.

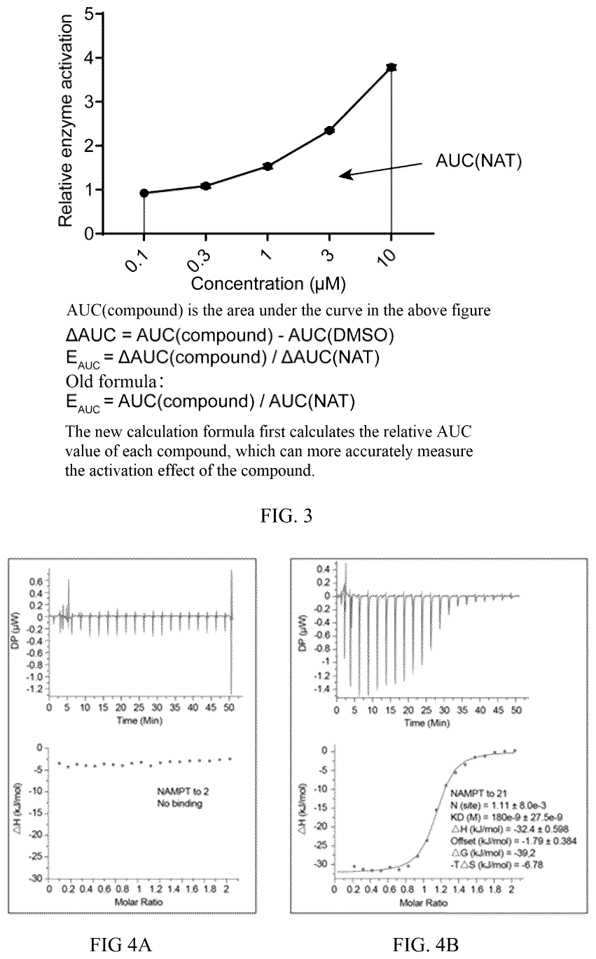

In the former assay, the compounds were added to the reaction solution (50mM Tris-HCl (pH 28.0), 12 mM MgCl, 1.5% ethanol, 15 μM PRPP, 2.5 mM ATP, 10mM semicarbazide, 0.2% BSA, 2.4 μg/ml NMNAT, 60 units of ADH, and 1 μM NAMPT) at concentrations of 0.1, 0.3, 1, 3 and 10 μM. The reaction was initiated by adding 200 μM nicotinamide (NAM) and mixing gently. The enzyme activity of NAMPT was expressed as the concentration of NADH generated per minute (the molar value was equal to NAD). The relative enzyme activity of NAMPT in each compound-treated group was normalized by the value of the DMSO-treated group, and a dose-response curve was drawn to evaluate the effect of individual compounds on NAMPT enzyme activity. The area under the dose-response curve (AUC, area under curve) was then calculated for individual compound and compared to the AUC of NAT, so that the obtained relative value quantitative E_{auc} represented the enzyme activation activity of each compound, and the specific calculation formula was shown in FIG. 3.

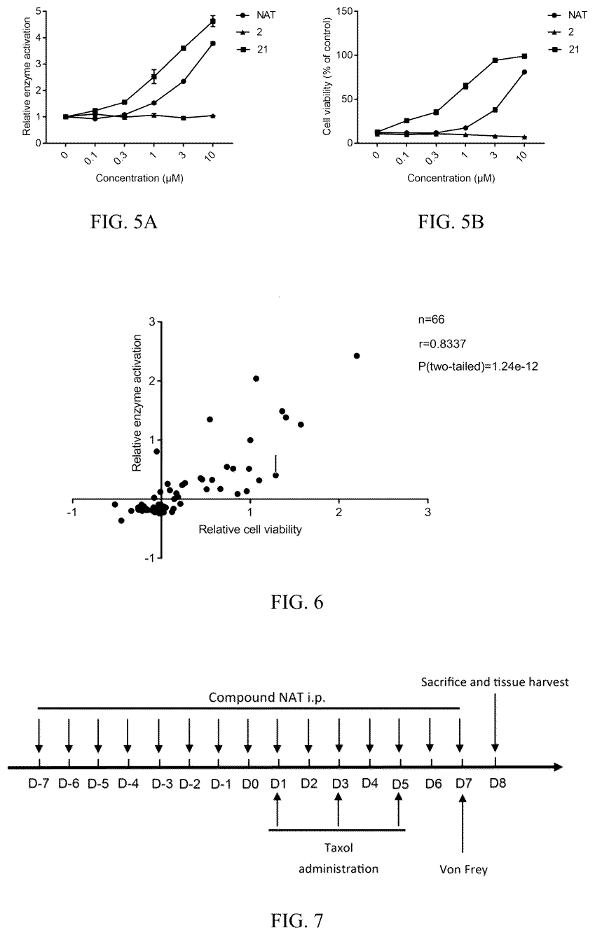

In the latter cell-based assay, we determined the degree of protection of NAT and derivatives thereof against the NAMPT inhibitor FK866. Individual compounds were added to the wells at final concentrations of 0.1, 0.3, 1, 3, and 10 μM, and 2 hours later all wells were treated with FK866 at a final concentration of 10 nM. After 72 hours, Celltiter-Glo (Promega) was used to detect cellular ATP levels to reflect cell viability and normalized to the DMSO group. A dose-response curve was drawn to evaluate the cytoprotective activity of individual compounds against FK866. Then the area under the dose-response curve (AUC, area under curve) of each compound was calculated and compared with the AUC of NAT, so that the obtained relative value quantitatively expressed the cytoprotective activity of each compound. Results for all compounds in both assays were summarized in Table 1. As shown in FIGS. 4A and 4B and FIGS. 5A and 5B, the dissociation constant Kd of compound 21 and NAMPT was about 180 nM, the binding affinity was stronger than that of NAT, and its activating enzyme activity and cytoprotective activity were also significantly improved compared with NAT; while compound 2 cannot bind NAMPT, and thus cannot activate NAMPT, nor protect cells. The structure-activity relationship study and the correlation analysis showed that the NAMPT activation activity of NAT and derivatives thereof was positively correlated with the cytoprotective activity, with a Pearson correlation coefficient r=0.83 (FIG. 6). These activities appeared to be determined by the affinity of the compound for NAMPT.

4. Neuroprotective Effect of NAT in Mouse Model of Chemotherapy Drug-Induced Peripheral Neuropathy

Chemotherapy-induced peripheral neuropathy (CIPN) was peripheral nerve damage resulted from anticancer chemotherapy, causing patients to experience persistent and progressive symptoms, including pain, numbness, tingling, and chills in the hands and feet. Chemotherapeutic drugs related to CIPN, such as paclitaxel and vinblastine, were widely used for anticancer treatment. Statistically, about 30-40% of patients receiving chemotherapy had symptoms of CIPN, but there was still no effective treatment drug (Y. Fukuda, Y. Li, R. A. Segal, A mechanistic understanding of axon degeneration in chemotherapy-induced peripheral neuropathy. Front Neurosci 11, 481 (2017).).

Using CIPN as an example of a neurodegenerative disease, we determined the neuroprotective activity of NAT in vivo. We established a mouse model of severe CIPN as shown in FIG. 7.

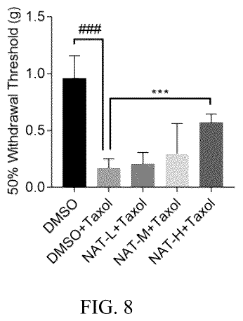

The first day of paclitaxel injection was taken as D1, and NAT was administered one week in advance (D-7) until D7, each group of 6 mice was injected with NAT at doses of 0, 3, 10 and 30 mg/kg every day. One week after NAT was administered in advance, paclitaxel was administered at a dose of 18.3 mg/kg every other day starting at D0, and the fiber needle mechanical prick test was performed on the second day (D7) after the last administration of paclitaxel (D5). The results showed that NAT administration at 30 mg/kg/day could significantly increase the mouse paw sting threshold in the mouse model of CIPN (as shown in FIG. 8).

TABLE 1
Activation of NAMPT activity and cytoprotective activity of NAT and
derivatives thereof*

| | | Relative activation | Relative cytoprotective |
|---|---|---|---|
| Compound No. | Structure | of NAMPT activity | activity |
| 1 | 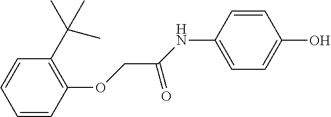 [SMILES: CC(C)(C)c1ccccc1OCC(=O)Nc1ccc(O)cc1] | 1 | 1 |
| 2 | 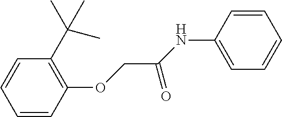 [SMILES: CC(C)(C)c1ccccc1OCC(=O)Nc1ccccc1] | −0.2247 | 0.02446 |
| 3 | 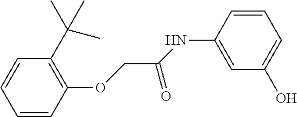 [SMILES: CC(C)(C)c1ccccc1OCC(=O)Nc1cccc(O)c1] | −0.1895 | −0.1032 |
| 4 | 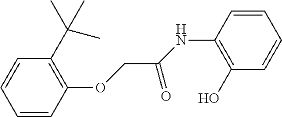 [SMILES: CC(C)(C)c1ccccc1OCC(=O)Nc1ccccc1O] | −0.1833 | −0.172 |
| 5 | 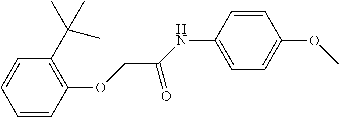 [SMILES: COc1ccc(NC(=O)COc2ccccc2C(C)(C)C)cc1] | −0.168 | −0.1713 |
| 6 | 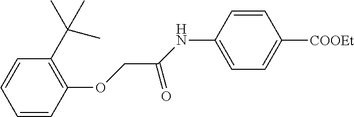 [SMILES: CCOC(=O)c1ccc(NC(=O)COc2ccccc2C(C)(C)C)cc1] | −0.1512 | −0.241 |
| 7 | 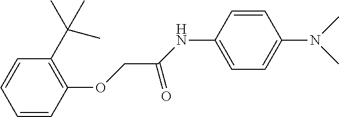 [SMILES: CN(C)c1ccc(NC(=O)COc2ccccc2C(C)(C)C)cc1] | −0.1777 | −0.2468 |
| 8 | 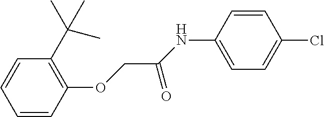 [SMILES: CC(C)(C)c1ccccc1OCC(=O)Nc1ccc(Cl)cc1] | −0.1983 | −0.3405 |
| 9 | 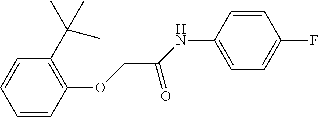 [SMILES: CC(C)(C)c1ccccc1OCC(=O)Nc1ccc(F)cc1] | −0.2123 | −0.0828 |
| 10 | 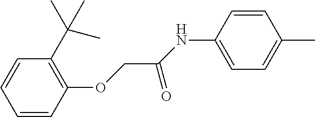 [SMILES: Cc1ccc(NC(=O)COc2ccccc2C(C)(C)C)cc1] | −0.1608 | −0.0679 |
| 11 | 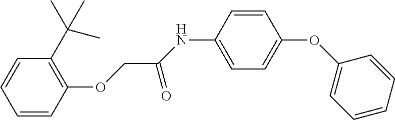 [SMILES: CC(C)(C)c1ccccc1OCC(=O)Nc1ccc(Oc2ccccc2)cc1] | −0.1409 | −0.0906 |
| 12 | 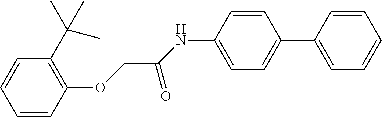 [SMILES: CC(C)(C)c1ccccc1OCC(=O)Nc1ccc(-c2ccccc2)cc1] | −0.0947 | −0.2237 |
| 13 | 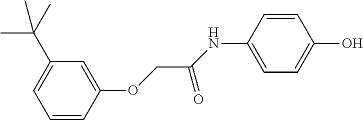 [SMILES: CC(C)(C)c1cccc(OCC(=O)Nc2ccc(O)cc2)c1] | 0.27328 | 0.26533 |
| 14 | 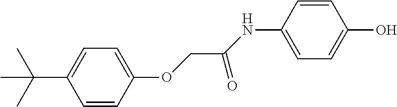 [SMILES: CC(C)(C)c1ccc(OCC(=O)Nc2ccc(O)cc2)cc1] | −0.1236 | 0.01994 |
| 15 | 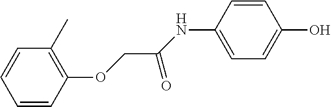 [SMILES: Cc1ccccc1OCC(=O)Nc1ccc(O)cc1] | 0.35503 | 0.44017 |
| 16 | 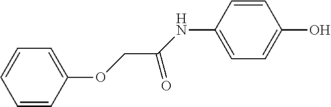 [SMILES: O=C(COc1ccccc1)Nc1ccc(O)cc1] | 0.0971 | 0.16839 |
| 17 | 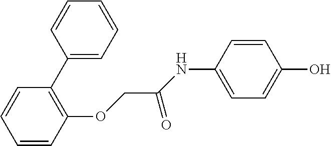 [SMILES: O=C(COc1ccccc1-c1ccccc1)Nc1ccc(O)cc1] | 0.33535 | 0.45719 |
| 18 | 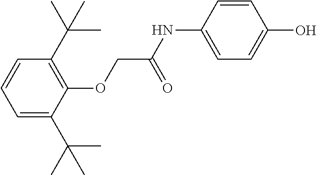 [SMILES: CC(C)(C)c1cccc(C(C)(C)C)c1OCC(=O)Nc1ccc(O)cc1] | −0.142 | 0.04793 |
| 19 | 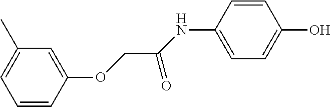 [SMILES: Cc1cccc(OCC(=O)Nc2ccc(O)cc2)c1] | 0.15065 | 0.09517 |
| 20 | 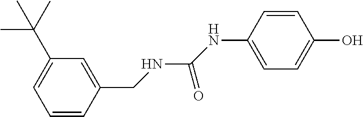 [SMILES: CC(C)(C)c1cccc(CNC(=O)Nc2ccc(O)cc2)c1] | −0.1622 | 0.13497 |
| 21 | 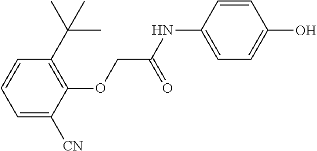 [SMILES: CC(C)(C)c1cccc(C#N)c1OCC(=O)Nc1ccc(O)cc1] | 2.42548 | 2.19792 |
| 22 | 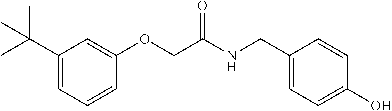 [SMILES: CC(C)(C)c1cccc(OCC(=O)NCc2ccc(O)cc2)c1] | −0.2162 | 0.11869 |
| 23 | 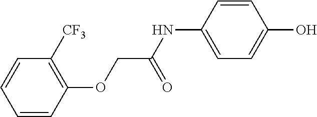 [SMILES: O=C(COc1ccccc1C(F)(F)F)Nc1ccc(O)cc1] | 0.23887 | 0.23373 |
| 24 | 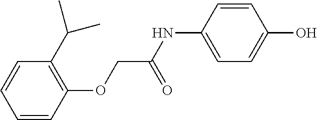 [SMILES: CC(C)c1ccccc1OCC(=O)Nc1ccc(O)cc1] | 1.35036 | 0.54472 |
| 25 | 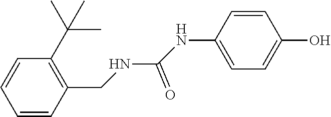 [SMILES: CC(C)(C)c1ccccc1CNC(=O)Nc1ccc(O)cc1] | −0.2226 | −0.0021 |
| 26 | 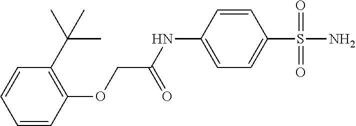 [SMILES: CC(C)(C)c1ccccc1OCC(=O)Nc1ccc(S(N)(=O)=O)cc1] | −0.2346 | −0.0055 |
| 27 | 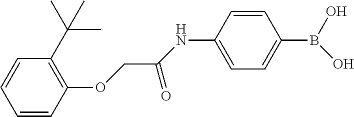 [SMILES: CC(C)(C)c1ccccc1OCC(=O)Nc1ccc(B(O)O)cc1] | 0.12174 | −0.0126 |
| 28 | 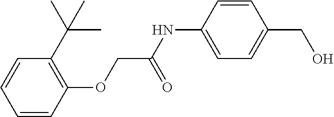 [SMILES: CC(C)(C)c1ccccc1OCC(=O)Nc1ccc(CO)cc1] | −0.2198 | −0.0697 |
| 29 | 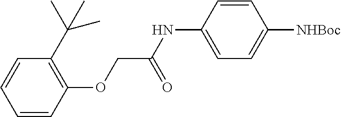 [SMILES: CC(C)(C)OC(=O)Nc1ccc(NC(=O)COc2ccccc2C(C)(C)C)cc1] | −0.1978 | −0.09 |
| 30 | 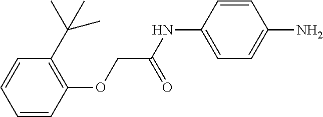 [SMILES: CC(C)(C)c1ccccc1OCC(=O)Nc1ccc(N)cc1] | 0.80749 | −0.0536 |
| 31 | 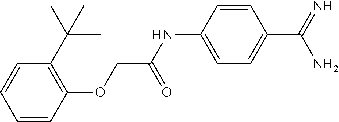 [SMILES: CC(C)(C)c1ccccc1OCC(=O)Nc1ccc(C(=N)N)cc1] | −0.1684 | −0.0343 |
| 32 | 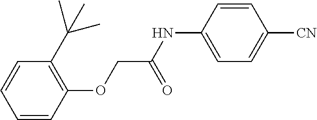 [SMILES: CC(C)(C)c1ccccc1OCC(=O)Nc1ccc(C#N)cc1] | −0.2396 | −0.0243 |
| 33 | 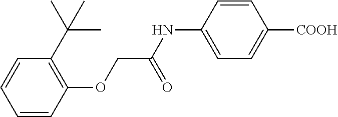 [SMILES: CC(C)(C)c1ccccc1OCC(=O)Nc1ccc(C(=O)O)cc1] | −0.1619 | −0.0221 |
| 34 | 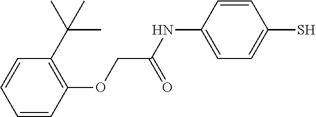 [SMILES: CC(C)(C)c1ccccc1OCC(=O)Nc1ccc(S)cc1] | −0.096 | −0.0072 |
| 35 | 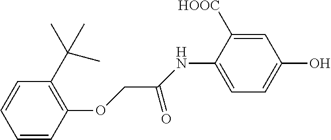 [SMILES: CC(C)(C)c1ccccc1OCC(=O)Nc1ccc(O)cc1C(=O)O] | −0.0889 | −0.5239 |
| 36 | 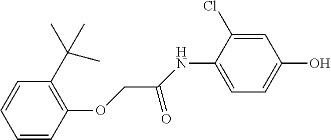 [SMILES: CC(C)(C)c1ccccc1OCC(=O)Nc1ccc(O)cc1Cl] | 0.25828 | 0.06922 |
| 37 | 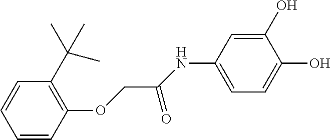 [SMILES: CC(C)(C)c1ccccc1OCC(=O)Nc1ccc(O)c(O)c1] | −0.1366 | −0.088 |
| 38 | 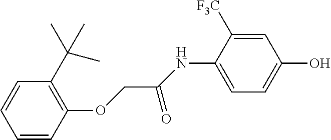 [SMILES: CC(C)(C)c1ccccc1OCC(=O)Nc1ccc(O)cc1C(F)(F)F] | −0.12 | −0.1996 |
| 39 | 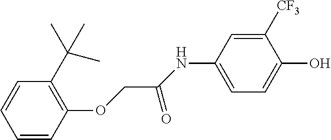 [SMILES: CC(C)(C)c1ccccc1OCC(=O)Nc1ccc(O)c(C(F)(F)F)c1] | −0.1435 | −0.2612 |
| 40 | 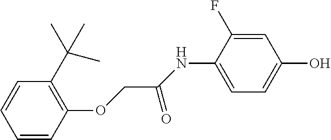 [SMILES: CC(C)(C)c1ccccc1OCC(=O)Nc1ccc(O)cc1F] | 0.03898 | 0.18586 |
| 41 | 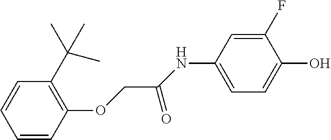 [SMILES: CC(C)(C)c1ccccc1OCC(=O)Nc1ccc(O)c(F)c1] | 0.13478 | 0.95879 |
| 42 | 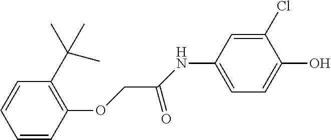 [SMILES: CC(C)(C)c1ccccc1OCC(=O)Nc1ccc(O)c(Cl)c1] | −0.1833 | −0.154 |
| 43 | 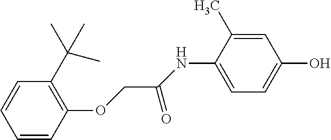 [SMILES: Cc1cc(O)ccc1NC(=O)COc1ccccc1C(C)(C)C] | −0.168 | −0.1826 |
| 44 | 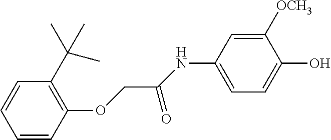 [SMILES: COc1cc(NC(=O)COc2ccccc2C(C)(C)C)ccc1O] | −0.1512 | −0.1872 |
| 45 | 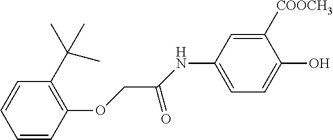 [SMILES: COC(=O)c1cc(NC(=O)COc2ccccc2C(C)(C)C)ccc1O] | −0.3625 | −0.4518 |
| 46 | 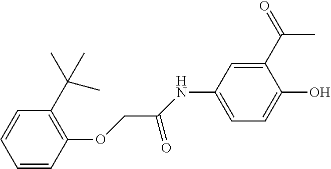 [SMILES: CC(=O)c1cc(NC(=O)COc2ccccc2C(C)(C)C)ccc1O] | −0.1777 | −0.2583 |
| 47 | 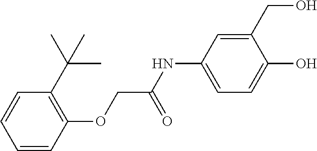 [SMILES: CC(C)(C)c1ccccc1OCC(=O)Nc1ccc(O)c(CO)c1] | −0.1983 | −0.2164 |
| 48 | 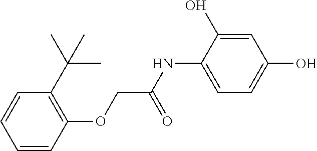 [SMILES: CC(C)(C)c1ccccc1OCC(=O)Nc1ccc(O)cc1O] | −0.1608 | −0.0776 |
| 49 | 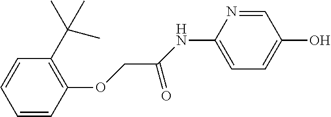 [SMILES: CC(C)(C)c1ccccc1OCC(=O)Nc1ccc(O)cn1] | −0.0947 | −0.032 |
| 50 | 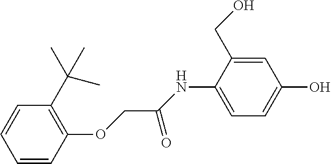 [SMILES: CC(C)(C)c1ccccc1OCC(=O)Nc1ccc(O)cc1CO] | −0.1356 | −0.0225 |
| 51 | 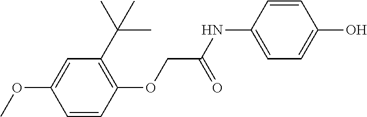 [SMILES: COc1ccc(OCC(=O)Nc2ccc(O)cc2)c(C(C)(C)C)c1] | 1.382 | 1.40157 |
| 52 | 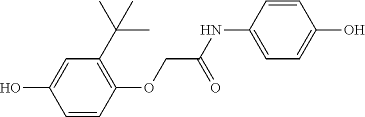 [SMILES: CC(C)(C)c1cc(O)ccc1OCC(=O)Nc1ccc(O)cc1] | 1.48841 | 1.35819 |
| 53 | 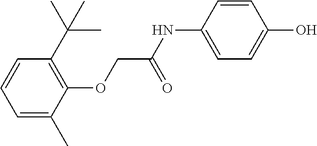 [SMILES: Cc1cccc(C(C)(C)C)c1OCC(=O)Nc1ccc(O)cc1] | 0.32704 | 0.57099 |
| 54 | 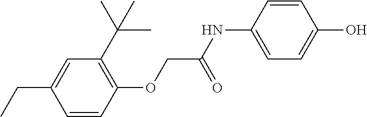 [SMILES: CCc1ccc(OCC(=O)Nc2ccc(O)cc2)c(C(C)(C)C)c1] | 0.08755 | 0.85539 |
| 55 | 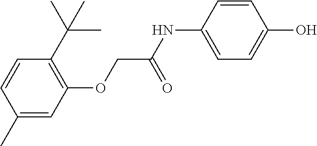 [SMILES: Cc1ccc(C(C)(C)C)c(OCC(=O)Nc2ccc(O)cc2)c1] | 0.17255 | 0.66228 |
| 56 | 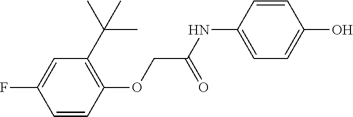 [SMILES: CC(C)(C)c1cc(F)ccc1OCC(=O)Nc1ccc(O)cc1] | 0.51283 | 0.98457 |
| 57 | 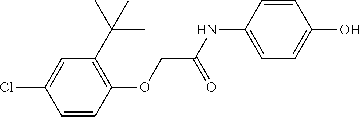 [SMILES: CC(C)(C)c1cc(Cl)ccc1OCC(=O)Nc1ccc(O)cc1] | 0.3183 | 1.09925 |
| 58 | 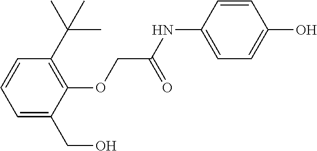 [SMILES: CC(C)(C)c1cccc(CO)c1OCC(=O)Nc1ccc(O)cc1] | 0.51589 | 0.80713 |
| 59 | 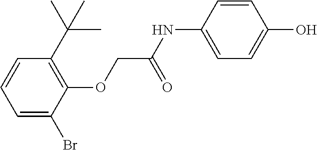 [SMILES: CC(C)(C)c1cccc(Br)c1OCC(=O)Nc1ccc(O)cc1] | 0.16811 | 0.50848 |
| 60 | 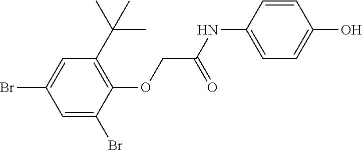 [SMILES: CC(C)(C)c1cc(Br)cc(Br)c1OCC(=O)Nc1ccc(O)cc1] | 0.02456 | −0.0824 |
| 61 | 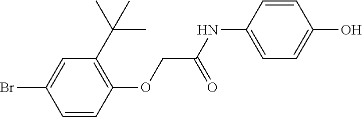 [SMILES: CC(C)(C)c1cc(Br)ccc1OCC(=O)Nc1ccc(O)cc1] | 0.40316 | 1.28494 |
| 62 | 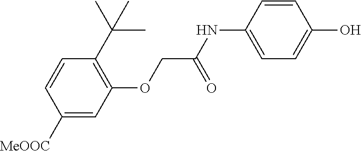 [SMILES: COC(=O)c1ccc(C(C)(C)C)c(OCC(=O)Nc2ccc(O)cc2)c1] | 0.54645 | 0.73592 |
| 63 | 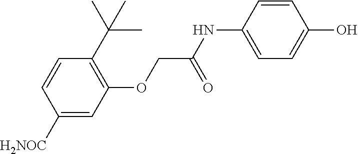 [SMILES: CC(C)(C)c1ccc(C(N)=O)cc1OCC(=O)Nc1ccc(O)cc1] | 2.04101 | 1.06536 |
| 64 | 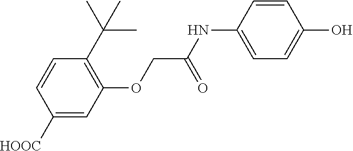 [SMILES: CC(C)(C)c1ccc(C(=O)O)cc1OCC(=O)Nc1ccc(O)cc1] | −0.0799 | 0.2131 |
| 65 | 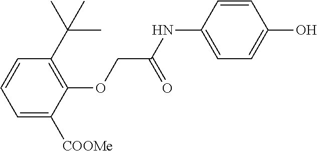 [SMILES: COC(=O)c1cccc(C(C)(C)C)c1OCC(=O)Nc1ccc(O)cc1] | 0.00292 | 0.14306 |
| 66 | 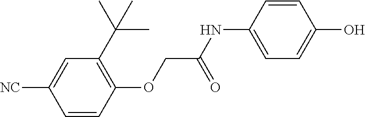 [SMILES: CC(C)(C)c1cc(C#N)ccc1OCC(=O)Nc1ccc(O)cc1] | 1.2627 | 1.56878 |

*The specific calculation method of relative cytoprotective activity and relative activation of NAMPT activity of NAT and derivatives thereof was shown in FIG. 3.

### INDUSTRIAL UTILITY

The present invention screens the NAMPT agonist NAT from the chemical small molecule library, and the NAT exhibits a good cytoprotective effect and a good anti-neurodegeneration effect in animal models of neurodegeneration. We studied the binding of NAT to enzymes, and then carried out multiple rounds of structure optimization based on the chemical structure characteristics of NAT and its enzyme activation properties, and obtained a relatively defined structure-activity relationship. The present patent not only lays the foundation for developing innovative drugs for anti-aging and neurodegenerative diseases, but also theoretically provides a proof-of-concept that enhancing NAMPT enzyme activity plays an important role in neuroprotection.

1-10. (canceled)

11. An aromatic compound having structural formula shown in formula I or formula 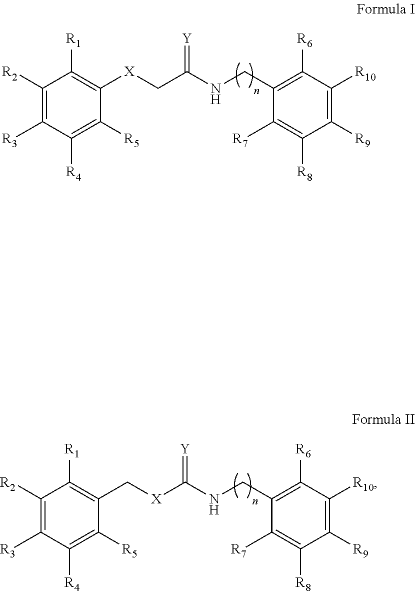
where, X represents 0 or NH;
Y represents O;
n is 0 or 1;
R₁, R₂, R₃, R₄, R₅, R₆, R₇, R₈, R₉, R₁₀ each independently represent H, C1-C6 straight or branched chain alkyl, C3-C6 cycloalkyl, halogen substituted C1-C6 straight or branched chain alkyl, hydroxyl, mercapto, halogen, cyano, nitro, boronic acid group, boron ester group, carboxyl, ester, carbonyl, phenoxy, amidino, amido, imide, sulfanilamide, pyrazolyl, substituted or unsubstituted amino, substituted or unsubstituted morpholinyl, substituted or unsubstituted piperidyl, substituted or unsubstituted piperazinyl, substituted or unsubstituted phenyl, substituted or unsubstituted pyridyl, substituted or unsubstituted C1-C6 alkoxy, substituted or unsubstituted C1-C4 alkyl hydroxyl, substituted or unsubstituted C1-C4 alkyl morpholinyl, substituted or unsubstituted C1-C4 alkyl piperidinyl, substituted or unsubstituted C1-C4 alkyl piperazinyl;
the substituted morpholinyl means that one or more carbons on the morpholinyl are substituted by the following groups: hydroxyl, C1-C6 alkyl, C1-C6 alkoxy, halogen, halogen substituted C1-C6 alkyl (such as trifluoromethyl), nitro, cyano, amino or substituted amino;
the substituted piperidinyl means that one or more carbons on the piperidinyl are substituted by the following groups: hydroxyl, C1-C6 alkyl, C1-C6 alkoxy, halogen, halogen substituted C1-C6 alkyl (such as trifluoromethyl), nitro, cyano, amino or substituted amino;
the substituted piperazinyl means that one or more carbons on the piperazinyl are substituted by the following groups: hydroxyl, C1-C6 alkyl, C1-C6 alkoxy, halogen, halogen substituted C1-C6 alkyl (such as trifluoromethyl), nitro, cyano, amino or substituted amino, and can also be that the H on the N of piperazinyl is substituted by the following groups:
unsubstituted C1-C6 alkyl or substituted C1-C6 alkyl, unsubstituted C1-C6 alkoxy or substituted C1-C6 alkoxy, acyl;
the substituted C1-C6 alkyl means that one or more hydrogens on the unsubstituted C1-C6 alkyl are substituted by hydroxyl, halogen, nitro, cyano, amino, unsubstituted phenyl or substituted phenyl;
the unsubstituted C1-C4 alkoxy is selected from a group consisting of methoxy, ethoxy, n-propoxy, isopropoxy, n-butoxy, isobutoxy, or tert-butoxy; the substituted C1-C4 alkoxy means that one or more hydrogens on the unsubstituted C1-C4 alkoxy are substituted by hydroxyl, halogen, nitro, cyano, amino, phenyl or substituted phenyl, and can also mean that one or more carbons on the unsubstituted C1-C4 alkoxy are substituted by O, N;
the substituted phenyl means that one or more hydrogens on the benzene ring are substituted by the following groups: hydroxyl, unsubstituted C1-C4 alkyl or substituted C1-C4 alkyl, unsubstituted C1-C4 alkoxy or substituted C1-C4 alkoxy, halogen, nitro, cyano, amino;
the unsubstituted pyridyl means that the connection position is on different carbons of the pyridyl, such as 2-pyridyl, 3-pyridyl, 4-pyridyl; the substituted pyridyl means that one or more carbons on the pyridyl are substituted by the following groups: hydroxyl, C1-C4 alkyl, C1-C4 alkoxy, halogen, halogen substituted 1C-6C alkyl (such as trifluoromethyl), nitro, cyano, amino or substituted amino;
the unsubstituted C1-C4 alkylamino is selected from a group consisting of methylamino, ethylamino, n-propylamino, isopropylamino, formylamino, acetamido, formimidoamino, cyclopropylamino, cyclobutylamino, cyclopentylamino, cyclohexylamino; the substituted C1-C4 alkylamino means that one or more hydrogens on the alkyl of unsubstituted C1-C4 alkylamino are substituted by hydroxyl, halogen, cyano, amino, phenyl or substituted phenyl, and can also mean that one or more carbons on the alkyl of unsubstituted C1-C4 alkylamino are substituted by N, and can also mean that one or more H on N of the unsubstituted C1-C4 alkylamino is substituted by methyl, ethyl, formyl, acetyl, cyclopropylamino, cyclobutylamino, cyclopentylamino, cyclohexylamino;
the unsubstituted C1-C4 alkyl hydroxyl means methyl hydroxyl, ethyl hydroxyl, n-propyl hydroxyl, isopropyl hydroxyl, n-butyl hydroxyl, isobutyl hydroxyl, tert-butyl hydroxyl; the substituted C1-C4 alkyl hydroxyl means that one or more hydrogens on the alkyl of unsubstituted C1-C4 alkyl hydroxyl are substituted by hydroxyl, halogen, cyano, amino, phenyl or substituted phenyl, and can also means that one or more carbons on the alkyl of the unsubstituted C1-C4 alkyl hydroxyl are replaced by O, N;
the unsubstituted C1-C4 alkyl morpholinyl means that methyl morpholinyl, ethyl morpholinyl, n-propyl morpholinyl, isopropyl morpholinyl, n-butyl morpholinyl, isobutyl morpholinyl, tert-butyl morpholinyl; the substituted C1-C4 alkyl morpholinyl means that one or more hydrogens on the alkyl of the unsubstituted C1-C4 alkyl morpholinyl are substituted by hydroxyl, halogen, cyano, amino, phenyl or substituted phenyl, and can also mean that one or more carbons on the alkyl of unsubstituted C1-C4 alkyl morpholinyl are substituted by O, N;
the unsubstituted C1-C4 alkyl piperidinyl means that methyl piperidinyl, ethyl piperidinyl, n-propyl piperidinyl, isopropyl piperidinyl, n-butyl piperidinyl, isobutyl piperidinyl, tert-butyl piperidinyl; the substituted C1-C4 alkyl piperidinyl means that one or more hydrogens on the alkyl of unsubstituted C1-C4 alkyl piperidinyl are substituted by hydroxyl, halogen, cyano, amino, phenyl or substituted phenyl;
the unsubstituted C1-C4 alkyl piperazinyl means that methyl piperazinyl, ethyl piperazinyl, n-propyl piperazinyl, isopropyl piperazinyl, n-butyl piperazinyl, isobutyl piperazinyl, tert-butyl piperazinyl; the substituted C1-C4 alkyl piperazinyl means that one or more hydrogens on the alkyl of unsubstituted C1-C4 alkyl piperazinyl are replaced by hydroxyl, halogen, cyano, amino, phenyl or substituted phenyl, and can also mean that the H on the N of unsubstituted C1-C4 alkyl piperazinyl are substituted by the following groups: unsubstituted C1-C4 alkyl or substituted C1-C4 alkyl, unsubstituted C1-C4 alkoxy or substituted C1-C4 alkoxy, acyl;
the substituted amino is selected from a group consisting of methylamino, dimethylamino, ethylamino, diethylamino, n-propylamino, di-n-propylamino, isopropylamino, diisopropylamino, formylamino, acetylamino, formimidoamino, cyclopropylamino, cyclobutylamino, cyclopentylamino, cyclohexylamino, and can also mean that azacyclobutyl, azacyclopentyl, azacyclohexyl, 2-oxo-azacyclobutyl, 2-oxo-azacyclopentyl, 2-oxo-azacyclohexyl;
the benzene ring in formula I or formula II can also be substituted by other aromatic rings;
wherein, the substituted amino means that at least one H on the amino is substituted by C1-C6 alkyl or tert-butyloxycarboryl;
the other aromatic rings are pyridine rings, naphthalene rings, furan rings, pyrrole rings or quinoline rings;
or a pharmaceutically acceptable salt thereof.

12. Any of the following methods:
(A) a method for the synthesis of the compound shown in formula I of claim 11, comprising the following steps:
(1) reacting the compound shown in formula III with tert-butyl bromoacetate to obtain compound shown in formula IV, followed by the compound shown in formula IV in the presence of trifluoroacetic acid to obtain the compound shown in formula V; 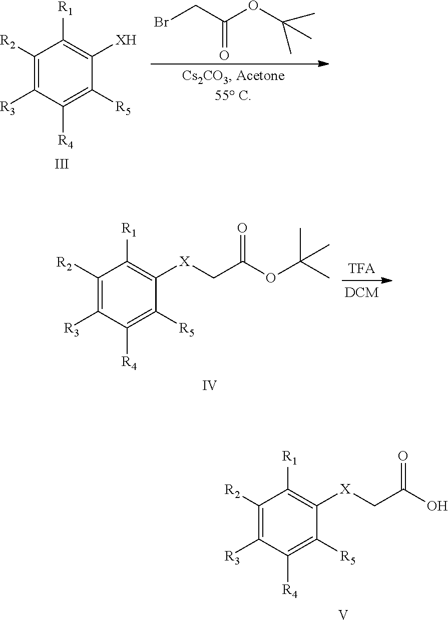
wherein the definitions of R₁, R₂, R₃, R₄, R₅, X in formulas III, IV and V are the same as the definitions of R₁, R₂, R₃, R₄, R₅, X in formula I of claim 11;
(2a) reacting the compound shown in formula V with oxalyl chloride to obtain the compound shown in formula VI, and then reacting the compound shown in formula VI with the compound shown in formula VII to obtain formula I; 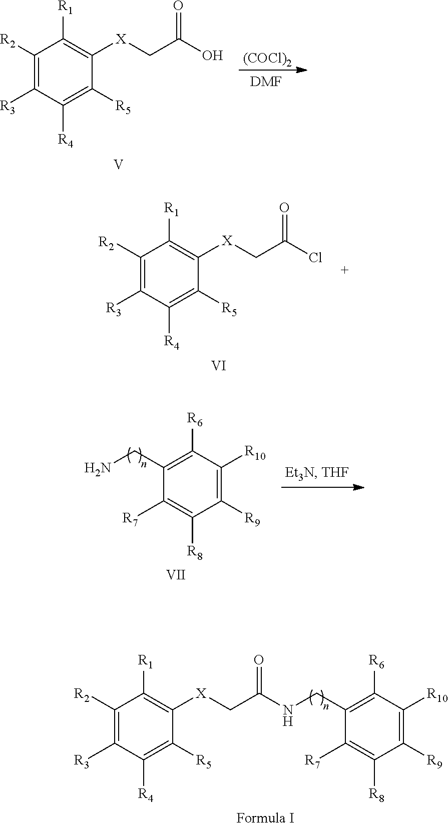
wherein the definitions of R₁, R₂, R₃, R₄, R₅, R₆, R₇, R₈, R₉, R₁₀, X, n in formula VI and VII are the same as the definitions of R₁, R₂, R₃, R₄, R₅, R₆, R₇, R₈, R₉, R₁₀, X, n in formula I of claim 11;
(B) a method for the synthesis of the compound shown in formula I of claim 11, comprising the following steps:
(1) reacting the compound shown in formula III with tert-butyl bromoacetate to obtain compound shown in formula IV, followed by the compound shown in formula IV in the presence of trifluoroacetic acid to obtain the compound shown in formula V; 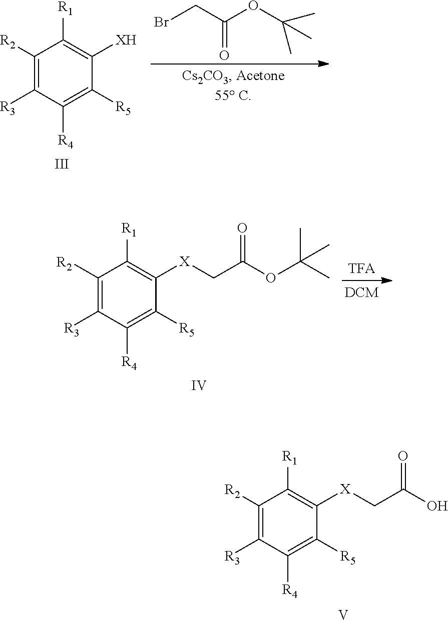
wherein the definitions of R₁, R₂, R₃, R₄, R₅, X in formulas III, IV and V are the same as the definitions of R₁, R₂, R₃, R₄, R₅, X in formula I of claim 11;
(2b) directly condensing formula V and formula VII under the conditions of 1-ethyl-(3-dimethylaminopropyl) carbodiimide hydrochloride and 1-hydroxybenzotriazole to obtain formula I; 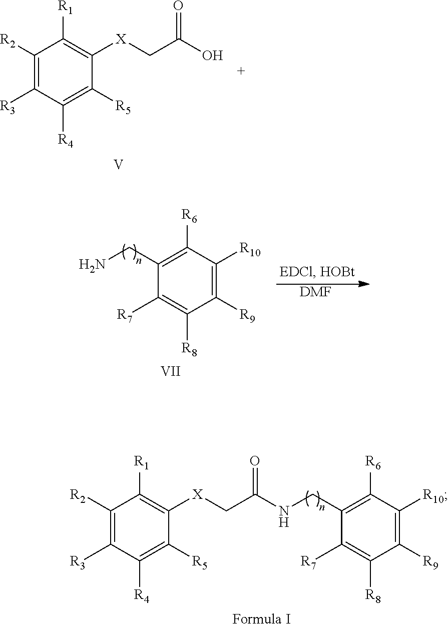
(C) a method for the synthesis of the compound shown in formula II of claim 11, comprising:
(1a) reacting formula X with formula XI to obtain formula XII, and then reacting formula XII with formula VIII to obtain formula II; 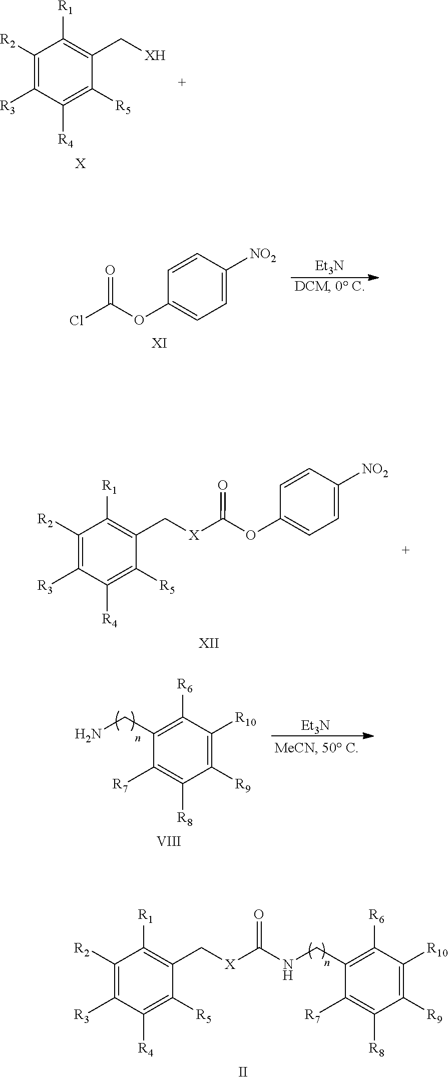
wherein the definitions of R₁, R₂, R₃, R₄, R₅, R₆, R₇, R₈, R₉, R₁₀, X in formula X, XI, XII are the same as the definitions of R₁, R₂, R₃, R₄, R₅, R₆, R₇, R₈, R₉, R₁₀, X in formula II of claim 11;
in VIII, the definitions of R₆, R₇, R₈, R₉, R₁₀ and n are the same as the definitions of R₆, R₇, R₈, R₉, R₁₀ and n in formula II of claim 11;
(D) a method for the synthesis of the compound shown in formula II of claim 11, comprising:
(1b) reacting formula X with formula XV to obtain formula II; 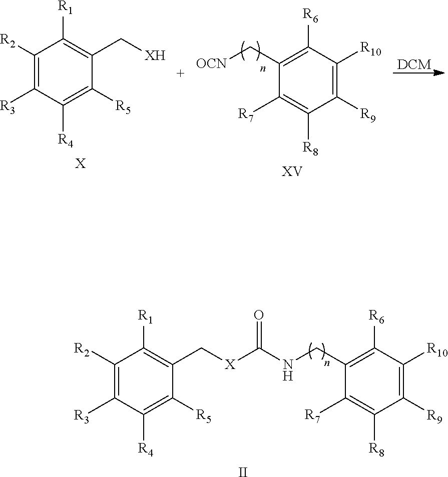
wherein the definitions of R₆, R₇, R₈, R₉, R₁₀, n in formula XV are the same as the definitions of R₆, R₇, R₈, R₉, R₁₀, n in formula II of claim 11;
(E) a method for preparing a NAMPT agonist;
(F) a method for anti-aging and treatment of neurodegenerative diseases.

13. The method according to claim 12, wherein the method for preparing a NAMPT agonist uses the aromatic compound represented by formula I or formula II or a pharmaceutically acceptable salt thereof.

14. The method according to claim 12, wherein the method for anti-aging and treatment of neurodegenerative diseases uses the aromatic compound represented by formula I or formula II or a pharmaceutically acceptable salt thereof.

15. Any of the following substances:
(G) a NAMPT agonist;
(H) a drug for treating neurodegenerative diseases or anti-aging.

16. The substance according to claim 15, wherein the active ingredient of the NAMPT agonist is the aromatic compound represented by formula I or formula II or a pharmaceutically acceptable salt thereof.

17. The substance according to claim 15, wherein the active ingredient of the drug for treating neurodegenerative diseases or anti-aging is the aromatic compound represented by formula I or formula II or a pharmaceutically acceptable salt thereof.
